(19) United States
(12) Patent Application Publication
(10) Pub. No.: US 2024/0026369 A1
(43) Pub. Date: Jan. 25, 2024

(54) USE OF ENHANCED POL THETA ACTIVITY FOR EUKARYOTIC GENOME ENGINEERING

(71) Applicant: KWS SAAT SE & Co. KGaA, Einbeck (DE)

(72) Inventor: Behailu AKLILU, Creve Coeur, MO (US)

(73) Assignee: KWS SAAT SE & Co. KGaA, Einbeck (DE)

(21) Appl. No.: 18/033,576

(22) PCT Filed: Oct. 26, 2021
(86) PCT No.: PCT/EP2021/079678

§ 371 Date: Apr. 25, 2023

Related U.S. Application Data
(60) Provisional application No. 63/106,104, filed on Oct. 27, 2020.

(51) Int. Cl. C12N 15/82; C12N 9/12; C12N 9/22

(52) U.S. Cl. CPC C12N 15/8213; C12N 9/1252; C12N 9/22; C12N 15/8218; C12N 2310/20

(57) ABSTRACT

The present invention generally relates to the technical field of targeted modification of a nucleotide sequence of interest in the genome of a plant by means of a site-specific nuclease, wherein the modification and precision is assisted by specifically enhancing DNA polymerase theta (Pol θ) activity to improve genome editing (GE) efficiencies, preferably for increasing targeted insertion of a DNA insert. The invention describes a method for increasing the efficiency of targeted transgene insertion in a plant or a plant cell. Further provided are methods to modulate endogenous repair pathways with the aim to favor Pol θ activity in the context of GE. Further disclosed are suitable sequences and GE tools suitable in the methods of the invention as well as cells, tissues, organs or materials obtainable by the methods. Finally, an expression construct assembly for conducting the methods of the invention is disclosed.

### TECHNICAL FIELD

The present invention generally relates to the technical field of targeted modification of a nucleotide sequence of interest in the genome of a plant by means of a site-specific nuclease, wherein the modification and precision is assisted by specifically enhancing DNA polymerase theta (Pol θ) activity to improve genome editing (GE) efficiencies, preferably for increasing targeted insertion of a DNA insert. The invention describes a method for increasing the efficiency of targeted transgene insertion in a plant or a plant cell. Further provided are methods to modulate endogenous repair pathways with the aim to favor Pol θ activity in the context of GE. Further disclosed are suitable sequences and GE tools suitable in the methods of the invention as well as cells, tissues, organs or materials obtainable by the methods. Finally, an expression construct assembly for conducting the methods of the invention is disclosed.

### BACKGROUND OF THE INVENTION

Meanwhile, the use of CRISPR-based genome editing (GE) is already described for various crop plants. Still, the efficiency of targeted transgene insertion is usually very poor (0.0-10%, Mao et al., National Science Review, Volume 6, Issue 3, May 2019, Pages 421-437, https://doi.org/10.1093/nsr/nwz005).

Most genome editing techniques rely on the introduction of targeted double strand breaks (DSB) by means of a site-specific nuclease, for example, a CRISPR system. The DSBs stimulate different cellular DNA repair pathways such as homology dependent repair (HDR, also often referred to as homologous recombination (HR)), non-homologous end-joining (NHEJ), and microhomology- or homology-mediated end-joining (MMEJ/HMEJ, see Yao et al., Cell Research, 27, 801-814 (2017)) resulting in targeted modifications (FIG. 1).

However, efficiency of targeted insertion remains largely poor (0.0-10%) calling for efforts to improve the performance of repair pathways for enhanced DNA knock-in capability.

NHEJ, a repair strategy not relying on any homologous regions, mostly causes random base pair insertions or deletions (InDels) that can lead to gene knockouts by disruption. In rarer cases, NHEJ repair mechanisms can also lead to the targeted introduction of a donor template if the repair template (RT) has fitting ends (blunt or sticky ends) that allow integration of the donor DNA.

HDR-based repair of the DSB can occur if a repair template is present that has matching regions to the target sequence (homologous arms) framing the sequence to be edited or inserted. Such HDR-based methods can create very precise insertions or base pair substitutions at the target site. This includes targeted insertion of larger nucleotide molecules, e.g. entire genes, or regulatory elements or the replacement of one allele for another, which for example carries exchange of one or more individual amino acid substitutions. The typical length of HDR-based repair homology arms is 50-1,000 nt (Yu et al, 2020: An efficient gene knock-in strategy using 5′-modified dsDNA donors with short homology arms; Li et al., 2019: Design and specificity of long ssDNA donors for CRISPR-based knock-in).

MMEJ and HMEJ pathways are both mediated by the DNA polymerase theta (Pol θ). Pol θ is a highly conserved, error prone, A-family DNA polymerase encoded by the PoIQ gene in multicellular eukaryotic organisms including plants (Yousefzadeh and Wood, DNA Repair, 12, 1-9, 2013; Sharief et al., Genomics, 59, 90-96, 1999). It contains an N-terminal helicase and a C-terminal polymerase domain connected by a long lesser conserved flexible region (Black et al., Molecular basis of microhomology-mediated end-joining by purified full-length Pol ⊖, Nature Communications, 10:4423, 2019). Pol θ plays role in DNA repair and genome integrity such as DNA interstrand crosslink and base excision repair (Pillaire et al., Oncogene (2010), 29, 876-887).

Most importantly, Pol θ is the main enzyme for a DSB repair pathway known by several names including “microhomology mediated end-joining (MMEJ)”, “alternative end-joining” (altEJ), and “polymerase theta-mediated end joining (TMEJ)” (Black et al., 2019, supra).

DNA polymerase theta (Polymerase theta, Pol theta, Pol Theta, or Pol θ herein with reference to the protein/enzyme), encoded by the POLQ gene (also PoIQ or Pol Q herein with reference to the DNA sequence) (e.g., for plants see: van Kregten et al., 2016, T-DNA integration in plants results from polymerase-θ-mediated DNA repair. Nature Plants 2, Article number: 16164), is a crucial enzyme involved in DNA repair in eukaryotic cells of various kingdoms.

Polymerase e was formerly considered a NHEJ component but has recently been implicated in microhomology-mediated end joining (MMEJ) processes, which are associated with insertions at the beak side (Sfeir and Symington, Trends in Biochemical Sciences, 40 (11), 701-714 (2015)).

DNA polymerase e in mammals is an a typical A-family type polymerase with an N-terminal helicase-like domain, a large central domain harboring a Rad51 interaction motif, and a C-terminal polymerase domain capable of extending DNA strands from mismatched or even unmatched termini. DNA molecules can be randomly incorporated into eukaryotic genomes through the action of Pol θ being a low fidelity polymerase (Hogg et al., 2012. Promiscuous DNA synthesis by human DNA polymerase e. Nucleic Acids Research, Volume 40, Issue 6, 1 Mar. 2012, Pages 2611-2622) that is required for random integration of T-DNAs in plants. Knockout mutant plants lacking Pol θ activity are incapable of integrating T-DNA molecules during Agrobacterium tumefaciens mediated plant transformation (van Kregten et al., 2016, supra). In vitro experiments identified an evolutionarily conserved loop in the polymerase domain that is essential for synapsing DNA ends during end joining to protect the genome against gross chromosomal rearrangements (Sfeir, The FASEB Journal, vol. 30, no. 1, 2016).

For MMEJ to occur in the natural cellular environment, two DSB ends have to be joined by Pol θ activity and these ends need to have homologous sequences that roughly range between 3 25 bp (Yanik et al., 2018; Sharma et al., 2015). The end ligation process starts with the resection of the 5′—strand ends to expose 3′—complementary single-stranded (ssDNA) homologous sequences or overhangs. The micro-homologous regions would then anneal followed by fill-in synthesis, ligation, and removal of heterologous flaps (Sfeir and Symington, 2015, supra). A report has also described activities of Pol θ on longer homology sequences (up to 1,600 nt) for the integration of transgenes in target DNA locus sites and named the mechanism “Homology-Mediated End joining (HMEJ)” (Yao et al., Cell Research, 2017, 27:801-814).

Meanwhile, several proteins were identified all of which are involved in the Pol θ mediated MMEJ repair pathway. In mammals, end resection is carried out by Mre11 and CtIP proteins (Truong et al., PNAS, 2013, vol. 110, no. 19). PARP1 is suggested to play the role of annealing microhomologies via complementary base pairing (Sfeir and Symington, 2015, supra).

Alternatively, Pol θ is proposed to anneal and tether broken DNA ends or stabilize annealed intermediates (Newman et al., 2015, Structure (12):2319-2330, doi: 10.1016/j.str.2015.10.014). Pol θ may also facilitate the displacement of ssDNA binding proteins such as RPA from the 3′-ssDNA overhangs. In addition, Pol θ is required for fill-in synthesis of ssDNA flanking the annealed microhomologies (Sfeir and Symington, 2015, supra). Finally, removal of heterologous flaps and sealing of ends is carried out by ERCC1/XPF and LIGASE3, respectively (Bennardo et al., 2008, PLoS Genetics, vol. 4, issue 6, el 000110; Simsek et al., 2011, PLoS Genetics, vol. 7, issue 6, e1002080).

Pol θ was also described to play a major role in the random integration of T-DNA into a plants' genome in its natural environment cellular (Kregten et al, 2016, supra), which findings explained the T-DNA insertion in a plant cell used in plant biotech applications since decades. No GE experiments were conducted though in Kregten et al, 2016, supra. In addition, Pol θ was disclosed as the primary facilitator of random integration of exogenous DNA in mammalian cells (Zelensky et al., 2017, Nature Communications, 8:66, PLoS Genetics, vol. 8, issue 2, e1002534), and retrohoming of linear group II introns (retrotransposons) in eukaryotes (White & Lambowitz, 2012).

Notably, Zelensky et al., 2017, supra, obtained the relevant results by Pol θ inactivation to decrease off-target random integration in a GE setting by hampering or abolishing Pol θ activity, which strategy aims at increasing HR targeting efficiencies. Interestingly, all groups and authors having worked with Pol θ in the context of GE exclusively tried to inactivate Pol θ and/or to abolish Pol θ activity in a cell to optimize GE outcomes as it was assumed that Pol θ activity may deteriorate targeted GE outcomes.

Although many efforts have been made to increase targeted and precise HDR-mediated gene editing to outcompete the more prevalent but error-prone NHEJ pathway, still at date little is known about increasing Pol θ-mediated repair pathways (MMEJ/NHEJ) to thereby increase targeted transgene insertion.

The present invention now surprisingly found that Pol θ activation can be exploited to significantly improve the outcome of GE for obtaining reliable targeted insertions and random insertions at a genomic target site of interest. This use of Pol θ as elucidated herein thus goes into the opposite direction of attempts disclosed in the prior art aiming at reducing or even abolishing Pol θ activity in a cell of interest during GE is conducted on this cell, particularly in plant cells.

It was thus an object of the present invention to optimize genome editing strategies in eukaryotic cells, preferably in plant cells, by increasing targeted or random insertion events by influencing cellular repair pathways in a manner favouring GE efficiency (rate of successful events) and specificity.

### SUMMARY OF THE INVENTION

The above objects were solved by technically exploiting the surprising finding that the activation, not the inhibition, of Pol θ in a cell of interest to be edited via GE can significantly enhance GE efficiency by providing new methods and molecular tools, and a combination thereof, specifically promoting Pol θ activity during GE.

In a first aspect, there is thus provided a method for targeted genome modification in a eukaryotic cell, preferably for increasing targeted insertion of a DNA insert at at least one target genomic sequence in a eukaryotic cell, comprising or consisting of the steps: a) providing at least one eukaryotic cell to be modified; b) promoting DNA polymerase theta (Pol θ) activity in said cell, wherein the promotion of Pol θ activity increases the DNA insertion efficiency during genome modification; c) introducing into the at least one eukaryotic cell (i) at least one genome modification system, preferably a genome editing system comprising at least one site-directed nuclease, nickase or an inactivated nuclease, preferably a nucleic acid guided nuclease, nickase or an inactivated nuclease, or a sequence encoding the same, and optionally at least one guide molecule, or a sequence encoding the same and (ii) at least one single-stranded or double-stranded DNA insert, or a sequence encoding the same; d) cultivating the at least one eukaryotic cell under conditions allowing (i) the promotion of DNA polymerase Pol θ (Pol θ) activity; and (ii) the activity of the at least one genome editing system and the at least one DNA insert and optionally the at least one guide molecule; and e) obtaining at least one modified cell comprising the sequence of the DNA insert integrated at or close to the target genomic site; and f) optionally: obtaining a eukaryotic organism, plant tissue, organ or seed regenerated from the at least one modified cell.

In one embodiment, the promoting of Pol θ is performed by enhancing the cellular level of polymerase Pol θ.

In a further embodiment of the above method, the DNA polymerase Pol θ sequence, or the POLQ sequence encoding the same, is selected from the group consisting of SEQ ID NO: 1 to SEQ ID NO: 26, or a sequence, including a functional domain, having at least at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity thereto.

In yet a further embodiment of the above method, the activity of DNA polymerase Pol θ is further promoted by modulating, preferably silencing, components of at least one competing DNA repair pathway, wherein the component belongs to the NHEJ or HDR repair pathway, or the sequence encoding the same, is selected from the group consisting of SEQ ID NO: 27 to SEQ ID NO: 114, or a sequence having at least at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity thereto.

In another embodiment of the above method, promotion of the MMEJ/HMEJ pathway is further achieved by co-expressing and/or modulating Pol θ and at least one MRE11 and/or at least one PARP1 protein, wherein the MRE11 and/or PARP1 protein, or the sequence encoding the same, is selected from the group consisting of SEQ ID NO: 115 to SEQ ID NO: 162 or a sequence having at least at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity thereto.

In yet a further embodiment of the above method, the at least one site-directed nuclease, nickase or inactivated nuclease, or a sequence encoding the same, is selected from the group consisting of a CRISPR/Cas system, preferably from a CRISPR/Cas12a or a CRISPR/Cas12b system, including a CRISPR/MAD7 system, a CRISPR/Cfp1 system, or a CRISPR/MAD2 system, from a CRISPR/Cas9 system, a CRISPR/CasX system, a CRISPR/CasY system, a CRISPR/Cas13 system, or a CRISPR/Csm system, a zinc finger nuclease system, a transcription activator-like nuclease system, or a meganuclease system, or any combination, variant, or catalytically active fragment thereof.

In another embodiment of the method according the first aspect, the Pol θ activity promotes targeted genome modification by acting on an endogenous micro-homology mediated end joining (MMEJ) and/or a homology-mediated end-joining (HMEJ) pathway.

In yet another embodiment of the method according the first aspect, the eukaryotic cell is a plant cell and wherein the method additionally comprises a step g) of screening for at least one plant tissue, organ, plant or seed regenerated from the at least or modified cell in the TO and/or T1 generation carrying the DNA insert.

In another embodiment of the method according the first aspect, the at least one eukaryotic cell is selected from a plant cell, or a mammalian cell, or the at least one eukaryotic cell is a plant cell, preferably wherein the plant cell is a plant cell selected from the group consisting of Hordeum, Sorghum, Saccharum, Zea, Setaria, Oryza, Triticum, Secale, Triticale, Malus, Brachypodium, Aegilops, Daucus, Beta, Eucalyptus, Nicotiana, Solanum, Coffea, Vitis, Erythrante, Genlisea, Cucumis, Marus, Arabidopsis, Crucihimalaya, Cardamine, Lepidium, Capsella, Olmarabidopsis, Arabis, Brassica, Eruca, Raphanus, Citrus, Jatropha, Populus, Medicago, Cicer, Cajanus, Phaseolus, Glycine, Gossypium, Astragalus, Lotus, Torenia, Allium, Spinacia or Helianthus, preferably, the plant or plant cell originates from a species selected from the group consisting of Hordeum vulgare, Hordeum bulbusom, Sorghum bicolor, Saccharum officinarium, Zea spp., including Zea mays, Setaria italica, Oryza minuta, Oryza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta spp., including Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Marus notabilis, Arabidopsis arenosa, Arabidopsis lyrata, Arabidopsis thaliana, Crucihimalaya himalaica, Crucihimalaya wallichii, Cardamine nexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Allium tuberosum, Helianthus annuus, Helianthus tuberosus and/or Spinacia oleracea.

In a further embodiment of the methods of the first aspect, the method additionally comprises the step of introducing or applying at least one regeneration booster, or a sequence encoding the same, or a regeneration booster chemical, preferably wherein the regeneration booster is selected from the group consisting of BBM, WUS, WOX, RKD4, RKD2, GRF, LEC, or a variant thereof, or a sequence encoding the same and/or wherein the regeneration booster comprises at least one of an RBP and/or at least one PLT, preferably wherein the regeneration booster comprises at least one of an RBP, wherein the at least one regeneration booster sequence is individually selected from any one of SEQ ID NOs: 171, 195 to 201, and 209 to 211 or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or an active fragment thereof, or wherein the at least one regeneration booster sequence is encoded by a sequence individually selected from any one of SEQ ID NO: 172, 202 to 208, and 212 to 214 or a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity thereto and/or wherein at least one first RBP or PLT, or a sequence encoding the same, preferably at least one RBP, most preferably RBP2, or the sequence encoding the same, is provided and wherein at least one further the regeneration booster is provided selected from: (i) at least one further RBP and/or PLT, or the sequence encoding the same, or a variant thereof, (ii) at least one BBM, or the sequence encoding the same, or a variant thereof, (iii) at least one WOX, including WUS1, WUS2, or WOX5, or the sequence encoding the same, or a variant thereof, (iv) at least one RKD4 or RKD2, including wheat RKD4, or the sequence encoding the same, or a variant thereof, (v) at least one GRF, including Zea mays GRF5 and Zea mays GRF1/TOW, or the sequence encoding the same, or a variant thereof, and/or (vi) at least one LEC sequence, including LEC1 and LEC2, or the sequence encoding the same, or a variant thereof, and wherein the at least one second regeneration booster, or a sequence encoding the same, is different to the first regeneration booster.

In a second aspect, there is provided a genetically modified cell, tissue, organ or material, including a seed, obtainable by a method according to the first aspect.

In one embodiment of the second aspect, the genetically modified cell, tissue, organ or material, including a seed, is a plant cell, tissue, organ or material, including a seed.

In a third aspect, there is provided an expression construct assembly, comprising: (i) at least one vector encoding at least one Pol Q sequence as defined above, and optionally comprising a construct encoding silencing components of at least one MMEJ/HMEJ competing repair pathway as defined above, and optionally a protein promoting MMEJ/HMEJ pathway as defined above; and (ii) at least one vector encoding a gene encoding at least one genome editing system, preferably wherein the genome editing system is as defined above, optionally comprising at least one vector encoding at least one guide molecule as defined in claim 1; and (iii) at least one vector encoding at least one DNA insert, or at least one single-stranded or double-stranded DNA insert as defined above; and (iv) optionally: at least one vector encoding at least one regeneration booster, preferably wherein the regeneration booster is as defined above; wherein (i), (ii), (iii), and/or (iv) are encoded on the same, or on different vectors.

In one embodiment of the third aspect, the at least one vector of the assembly further comprises a nucleic acid sequence encoding at least one marker.

In one embodiment of the third aspect, the expression construct assembly comprises at least one construct or sequence individually selected from any one of SEQ ID NOs: 163 to 165, 170, 173 to 175, and 179 to 183, 185, 187 and 189.

In yet other aspect, the methods as disclosed herein may be used in a therapeutic sense to use the surprising effect that Pol θ activity is associated with an increased GE efficiency if the competing endogenous DNA repair pathways are modified in a concerted way, to achieve at least one targeted genome modification in a eukaryotic cell, preferably a mammalian cell or a human cell, to modify a at least one mutation associated with a disease, wherein the method does not involve a method for treatment of the human or animal body by surgery or therapy and/or a diagnostic method practised on the human or animal body and/or wherein the method does not involve processes for cloning human beings, processes for modifying the germ line genetic identity of human beings, uses of human embryos for industrial or commercial purposes and/or processes for modifying the genetic identity of animals which are likely to cause them suffering without any substantial medical benefit to man or animal, and also animals resulting from such processes.

Further uses of and methods for constructing multiple purpose expression constructs and expression cassettes for use according to the present invention are provided.

### BRIEF DESCRIPTION OF DRAWINGS

FIG. 1 shows the major repair pathways as of relevance during gene editing and subsequent repair. Most genome editing techniques rely on the introduction of targeted double strand breaks (DSBs) by means of a site-specific nuclease, for example of the CRISPR system. The DSBs stimulate different cellular DNA repair pathways such as homology dependent repair (HDR, also often referred to as homologous recombination (HR)), non-homologous end-joining (NHEJ), or the Pol θ-mediated microhomology- or homology-mediated end-joining (MMEJ/HMEJ) resulting in targeted modifications. MMEJ is also often referred to as polymerase theta (Pol θ) mediated end-joining (TMEJ) or alternative end-joining (altEJ).

FIG. 2 shows % homology levels among Pol θ proteins based on their amino acid sequences (SEQ ID NOs 2, 4, 6, 8, 10, 12, 14, 16, 18, 20, 22, 24, 26). It should be noted that this comparison does not consider similarities between amino acid properties (e.g. charged, aromatic, hydrophobic). This percent identity matrix was created using clustal omega (htps://www.ebi ac.uk/Tools/nsa/clustalo/).

FIG. 3 shows a phylogenetic tree based on the Pol θ amino acid sequences. This tree was generated using the CLC Main Workbench 8.0.

FIG. 4 shows the exemplary DNA insert design for (A) MMEJ-mediated transgene insertion, and (B) HMEJ-mediated transgene insertion.

FIG. 5 shows the more detailed structure of the specific DNA insert used. *Plasmid seq is part of the plasmid backbone sequence included in the transgene as a result of an effort to pick good reverse primer for PCR amplification of the transgene.

FIG. 6 shows the sequence of the Zea mays Cell Wall Invertase (ZmCWI2) promoter (and portion of exon and intron) and the target cleavage sites. PAM sites are underlined, gRNA targets are boxed. The first exon is shaded in grey and the first intron in lower case. Cleavage occurs between the 18th and the 19th nucleotide (counting from the PAM site) of the gRNA sequence.

FIG. 7 shows a cartoon depicting the Zea mays Cell Wall Invertase (ZmCWI2) promoter (and portion of exon and intron) and target cleavage sites.

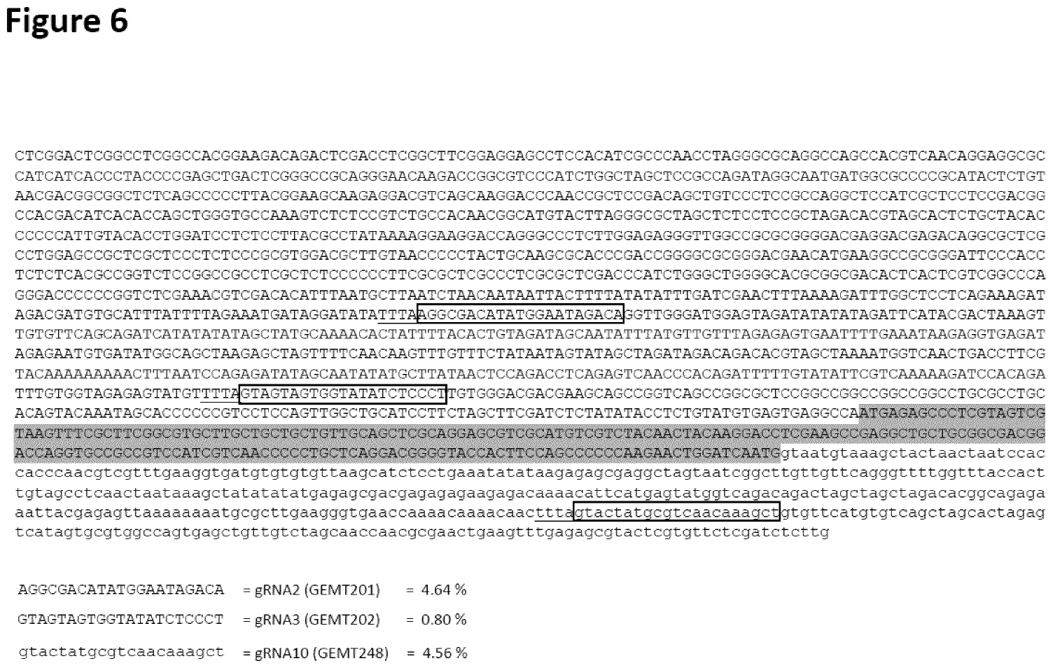

### BRIEF DESCRIPTION OF SEQUENCES

| SEQ ID NO. | Description |
|---|---|
| 1 | POLQ Beta Vulgaris (Sugar beet) cDNA sequence |
| 2 | Pol theta Beta Vulgaris (Sugar beet) amino acid sequence |
| 3 | POLQ Zea mays (corn) cDNA sequence |
| 4 | Pol theta Zea mays (corn) amino acid sequence |
| 5 | POLQ Brassica napus (Rapeseed or Oilseed rape) cDNA sequence |
| 6 | Pol theta Brassica napus (Rapeseed or Oilseed rape) amino acid sequence |
| 7 | POLQ Helianthus annus (Sunflower) cDNA sequence |
| 8 | Pol theta Helianthus annus (Sunflower) amino acid sequence |
| 9 | POLQ Hordeum vulgare (Barely) cDNA sequence |
| 10 | Pol theta Hordeum vulgare (Barely) amino acid sequence |
| 11 | POLQ Triticum aestivum (Wheat) cDNA sequence |
| 12 | Pol theta Triticum aestivum (Wheat) amino acid sequence |
| 13 | POLQ Secale cereale (Rye) cDNA sequence |
| 14 | Pol theta Secale cereale (Rye) amino acid sequence |
| 15 | POLQ Oryza Sativa (rice) cDNA sequence |
| 16 | Pol theta Oryza Sativa (rice) amino acid sequence |
| 17 | POLQ Glycine max (Soybean) cDNA sequence |
| 18 | Pol theta Glycine max (Soybean) amino acid sequence |
| 19 | POLQ Solanum tuberosum (Potato) cDNA sequence |
| 20 | Pol theta Solanum tuberosum (Potato) amino acid sequence |
| 21 | POLQ Solanum lycopersicum (Tomato) cDNA sequence |
| 22 | Pol theta Solanum lycopersicum (Tomato) amino acid sequence |
| 23 | POLQ Sorghum bicolor (Sorghum) cDNA sequence |
| 24 | Pol theta Sorghum bicolor (Sorghum) amino acid sequence |
| 25 | POLQ Homo sapiens (human) cDNA sequence |
| 26 | Pol theta Homo sapiens (human) amino acid sequence |
| 27 | RPA70C Beta Vulgaris (Sugar beet) cDNA sequence |
| 28 | RPA70C Beta Vulgaris (Sugar beet) amino acid sequence |
| 29 | RPA70C Zea mays (corn) cDNA sequence |
| 30 | RPA70C Zea mays (corn) amino acid sequence |
| 31 | RPA70C Brassica napus (Rapeseed or Oilseed rape) cDNA sequence |
| 32 | RPA70C Brassica napus (Rapeseed or Oilseed rape) amino acid sequence |
| 33 | RPA70C Helianthus annus (Sunflower) cDNA sequence |
| 34 | RPA70C Helianthus annus (Sunflower) amino acid sequence |
| 35 | RPA70C Hordeum vulgare (Barely) cDNA sequence |
| 36 | RPA70C Hordeum vulgare (Barely) amino acid sequence |
| 37 | RPA70C Triticum aestivum (Wheat) cDNA sequence |
| 38 | RPA70C Triticum aestivum (Wheat) amino acid sequence |
| 39 | RPA70C Secale cereale (Rye) cDNA sequence |
| 40 | RPA70C Secale cereale (Rye) amino acid sequence |
| 41 | RPA70C Oryza Sativa (rice) cDNA sequence |
| 42 | RPA70C Oryza Sativa (rice) amino acid sequence |
| 43 | RPA70C Glycine max (Soybean) cDNA sequence |
| 44 | RPA70C Glycine max (Soybean) amino acid sequence |
| 45 | RPA70C Solanum tuberosum (Potato) cDNA sequence |
| 46 | RPA70C Solanum tuberosum (Potato) amino acid sequence |
| 47 | RPA70C Solanum lycopersicum (Tomato) cDNA sequence |
| 48 | RPA70C Solanum lycopersicum (Tomato) amino acid sequence |
| 49 | RPA70C Sorghum bicolor (Sorghum) cDNA sequence |
| 50 | RPA70C Sorghum bicolor (Sorghum) amino acid sequence |
| 51 | DNA ligase 4 Beta Vulgaris (Sugar beet) cDNA sequence |
| 52 | DNA ligase 4 Beta Vulgaris (Sugar beet) amino acid sequence |
| 53 | DNA ligase 4 Zea mays (corn) cDNA sequence |
| 54 | DNA ligase 4 Zea mays (corn) amino acid sequence |
| 55 | DNA ligase 4 Brassica napus (Rapeseed or Oilseed rape) cDNA sequence |
| 56 | DNA ligase 4 Brassica napus (Rapeseed or Oilseed rape) amino acid sequence |
| 57 | DNA ligase 4 Helianthus annus (Sunflower) cDNA sequence |
| 58 | DNA ligase 4 Helianthus annus (Sunflower) amino acid sequence |
| 59 | DNA ligase 4 Hordeum vulgare (Barely) cDNA sequence |
| 60 | DNA ligase 4 Hordeum vulgare (Barely) amino acid sequence |
| 61 | DNA ligase 4 Secale cereale (Rye) cDNA sequence |
| 62 | DNA ligase 4 Secale cereale (Rye) amino acid sequence |
| 63 | DNA ligase 4 Oryza Sativa (rice) cDNA sequence |
| 64 | DNA ligase 4 Oryza Sativa (rice) amino acid sequence |
| 65 | DNA ligase 4 Glycine max (Soybean) cDNA sequence |
| 66 | DNA ligase 4 Glycine max (Soybean) amino acid sequence |
| 67 | DNA ligase 4 Solanum tuberosum (Potato) cDNA sequence |
| 68 | DNA ligase 4 Solanum tuberosum (Potato) amino acid sequence |
| 69 | DNA ligase 4 Solanum lycopersicum (Tomato) cDNA sequence |
| 70 | DNA ligase 4 Solanum lycopersicum (Tomato) amino acid sequence |
| 71 | DNA ligase 4 Sorghum bicolor (Sorghum) cDNA sequence |
| 72 | DNA ligase 4 Sorghum bicolor (Sorghum) amino acid sequence |
| 73 | KU70 Beta Vulgaris (Sugar beet) cDNA sequence |
| 74 | KU70 Beta Vulgaris (Sugar beet) amino acid sequence |
| 75 | KU70 Zea mays (corn) cDNA sequence |
| 76 | KU70 Zea mays (corn) amino acid sequence |
| 77 | KU70 Brassica napus (Rapeseed or Oilseed rape) cDNA sequence |
| 78 | KU70 Brassica napus (Rapeseed or Oilseed rape) amino acid sequence |
| 79 | KU70 Hordeum vulgare (Barely) cDNA sequence |
| 80 | KU70 Hordeum vulgare (Barely) amino acid sequence |
| 81 | KU70 Triticum aestivum (Wheat) cDNA sequence |
| 82 | KU70 Triticum aestivum (Wheat) amino acid sequence |
| 83 | KU70 Oryza Sativa (rice) cDNA sequence |
| 84 | KU70 Oryza Sativa (rice) amino acid sequence |
| 85 | KU70 Glycine max (Soybean) cDNA sequence |
| 86 | KU70 Glycine max (Soybean) amino acid sequence |
| 87 | KU70 Solanum tuberosum (Potato) cDNA sequence |
| 88 | KU70 Solanum tuberosum (Potato) amino acid sequence |
| 89 | KU70 Solanum lycopersicum (Tomato) cDNA sequence |
| 90 | KU70 Solanum lycopersicum (Tomato) amino acid sequence |
| 91 | KU70 Sorghum bicolor (Sorghum) cDNA sequence |
| 92 | KU70 Sorghum bicolor (Sorghum) amino acid sequence |
| 93 | KU80 Beta Vulgaris (Sugar beet) cDNA sequence |
| 94 | KU80 Beta Vulgaris (Sugar beet) amino acid sequence |
| 95 | KU80 Zea mays (corn) cDNA sequence |
| 96 | KU80 Zea mays (corn) amino acid sequence |
| 97 | KU80 Brassica napus (Rapeseed or Oilseed rape) cDNA sequence |
| 98 | KU80 Brassica napus (Rapeseed or Oilseed rape) amino acid sequence |
| 99 | KU80 Helianthus annus (Sunflower) cDNA sequence |
| 100 | KU80 Helianthus annus (Sunflower) amino acid sequence |
| 101 | KU80 Hordeum vulgare (Barely) cDNA sequence |
| 102 | KU80 Hordeum vulgare (Barely) amino acid sequence |
| 103 | KU80 Triticum aestivum (Wheat) cDNA sequence |
| 104 | KU80 Triticum aestivum (Wheat) amino acid sequence |
| 105 | KU80 Oryza Sativa (rice) cDNA sequence |
| 106 | KU80 Oryza Sativa (rice) amino acid sequence |
| 107 | KU80 Glycine max (Soybean) cDNA sequence |
| 108 | KU80 Glycine max (Soybean) amino acid sequence |
| 109 | KU80 Solanum tuberosum (Potato) cDNA sequence |
| 110 | KU80 Solanum tuberosum (Potato) amino acid sequence |
| 111 | KU80 Solanum lycopersicum (Tomato) cDNA sequence |
| 112 | KU80 Solanum lycopersicum (Tomato) amino acid sequence |
| 113 | KU80 Sorghum bicolor (Sorghum) cDNA sequence |
| 114 | KU80 Sorghum bicolor (Sorghum) amino acid sequence |
| 115 | MRE11 Beta Vulgaris (Sugar beet) cDNA sequence |
| 116 | MRE11 Beta Vulgaris (Sugar beet) amino acid sequence |
| 117 | MRE11 Zea mays (corn) cDNA sequence |
| 118 | MRE11 Zea mays (corn) amino acid sequence |
| 119 | MRE11 Brassica napus (Rapeseed or Oilseed rape) cDNA sequence |
| 120 | MRE11 Brassica napus (Rapeseed or Oilseed rape) amino acid sequence |
| 121 | MRE11 Helianthus annus (Sunflower) cDNA sequence |
| 122 | MRE11 Helianthus annus (Sunflower) amino acid sequence |
| 123 | MRE11 Hordeum vulgare (Barely) cDNA sequence |
| 124 | MRE11 Hordeum vulgare (Barely) amino acid sequence |
| 125 | MRE11 Triticum aestivum (Wheat) cDNA sequence |
| 126 | MRE11 Triticum aestivum (Wheat) amino acid sequence |
| 127 | MRE11 Secale cereale (Rye) cDNA sequence |
| 128 | MRE11 Secale cereale (Rye) amino acid sequence |
| 129 | MRE11 Oryza Sativa (rice) cDNA sequence |
| 130 | MRE11 Oryza Sativa (rice) amino acid sequence |
| 131 | MRE11 Glycine max (Soybean) cDNA sequence |
| 132 | MRE11 Glycine max (Soybean) amino acid sequence |
| 133 | MRE11 Solanum tuberosum (Potato) cDNA sequence |
| 134 | MRE11 Solanum tuberosum (Potato) amino acid sequence |
| 135 | MRE11 Solanum lycopersicum (Tomato) cDNA sequence |
| 136 | MRE11 Solanum lycopersicum (Tomato) amino acid sequence |
| 137 | MRE11 Sorghum bicolor (Sorghum) cDNA sequence |
| 138 | MRE11 Sorghum bicolor (Sorghum) amino acid sequence |
| 139 | PARP1 Beta Vulgaris (Sugar beet) cDNA sequence |
| 140 | PARP1 Beta Vulgaris (Sugar beet) amino acid sequence |
| 141 | PARP1 Zea mays (corn) cDNA sequence |
| 142 | PARP1 Zea mays (corn) amino acid sequence |
| 143 | PARP1 Brassica napus (Rapeseed or Oilseed rape) cDNA sequence |
| 144 | PARP1 Brassica napus (Rapeseed or Oilseed rape) amino acid sequence |
| 145 | PARP1 Helianthus annus (Sunflower) cDNA sequence |
| 146 | PARP1 Helianthus annus (Sunflower) amino acid sequence |
| 147 | PARP1 Hordeum vulgare (Barely) cDNA sequence |
| 148 | PARP1 Hordeum vulgare (Barely) amino acid sequence |
| 149 | PARP1 Triticum aestivum (Wheat) cDNA sequence |
| 150 | PARP1 Triticum aestivum (Wheat) amino acid sequence |
| 151 | PARP1 Secale cereale (Rye) cDNA sequence (partial) |
| 152 | PARP1 Secale cereale (Rye) amino acid sequence (partial) |
| 153 | PARP1 Oryza Sativa (rice) cDNA sequence |
| 154 | PARP1 Oryza Sativa (rice) amino acid sequence |
| 155 | PARP1 Glycine max (Soybean) cDNA sequence |
| 156 | PARP1 Glycine max (Soybean) amino acid sequence |
| 157 | PARP1 Solanum tuberosum (Potato) cDNA sequence |
| 158 | PARP1 Solanum tuberosum (Potato) amino acid sequence |
| 159 | PARP1 Solanum lycopersicum (Tomato) cDNA sequence |
| 160 | PARP1 Solanum lycopersicum (Tomato) amino acid sequence |
| 161 | PARP1 Sorghum bicolor (Sorghum) cDNA sequence |
| 162 | PARP1 Sorghum bicolor (Sorghum) amino acid sequence |
| 163 | A general example of a DNA fragment (ZmUbi) flanked by homology arms |
| | for targeted insertion at ZmCWI2 promoter region. |
| 164 | Transgene (ZmUbi) to be integrated (excl. Homology arms) |
| 165 | Plasmid constructs for the expression of Mad7 and tDTomato (GEMT129) |
| 166 | Mad7 CDS |
| 167 | MAD7 PRT |
| 168 | tdTomato CDS |
| 169 | tdTomato PRT |
| 170 | Plasmid constructs for the expression of RBP2 (GEMT128) |
| 171 | RBP2 CDS |
| 172 | RBP2 PRT |
| 173 | GEMT201, plasmid that encodes gRNA to target CWI2 promoter |
| 174 | GEMT202, plasmid that encodes gRNA to target CWI2 promoter |
| 175 | GEMT248, plasmid that encodes gRNA to target CWI2 promoter |
| 176 | gRNA from GEMT201 |
| 177 | gRNA from GEMT202 |
| 178 | gRNA from GEMT248 |
| 179 | Plasmid construct for the expression of the DNA insert (SEQ ID NO 163) |
| 180 | Plasmid construct expressing ZmPolQ (GMT242) |
| 181 | Plasmid construct GEMT341 expressing ZmPARP1 (SEQ ID NO: 141 |
| | (CDS) and 142 (PRT) |
| 182 | Plasmid construct GEMT340 expressing ZmMre11 (SEQ ID NO: 117 (CDS) |
| | and 118 (PRT) |
| 183 | Plasmid construct GEMT268 expressing a ZmRPA70C silencing construct |
| 184 | ZmRPA70C silencing construct |
| 185 | Plasmid construct GEMT269 expressing a ZmKu70 silencing construct |
| 186 | ZmKu70 silencing construct |
| 187 | Plasmid construct expressing a ZmKu80 silencing construct |
| 188 | ZmKu80 silencing construct |
| 189 | Plasmid construct GEMT270 expressing a ZmLig4 silencing construct |
| 190 | ZmLig4 silencing construct |
| 191 | FIG. 6 |
| 192 | FIG. 6 gRNA 2 |
| 193 | FIG. 6 gRNA 3 |
| 194 | FIG. 6 gRNA 10 |
| 195 | RBP1 CDS |
| 196 | RBP3 CDS |
| 197 | RBP4 CDS |
| 198 | RBP5 CDS |
| 199 | RBP6 CDS |
| 200 | RBP7 CDS |
| 201 | RBP8 CDS |
| 202 | RBP1 protein |
| 203 | RBP3 protein |
| 204 | RBP4 protein |
| 205 | RBP5 protein |
| 206 | RBP6 protein |
| 207 | RBP7 protein |
| 208 | RBP8 protein |
| 209 | ZmPLT3-17207_CDS |
| 210 | ZmPLTS_CDS |
| 211 | ZmPLT7_CDS |
| 212 | PLT3-17207 protein |
| 213 | ZmPLTS protein |
| 214 | ZmPLT7 protein |

### Definitions

Unless defined otherwise, technical and scientific terms used herein have the same meaning as commonly understood by one of ordinary skill in the art to which this invention belongs.

As used in the context of the present application, the term “about” means+/−10% of the recited value, preferably +/−5% of the recited value. For example, about 100 nucleotides (nt) shall be understood as a value between 90 and 110 nt, preferably between 95 and 105 nt.

A “base editor” as used herein refers to a protein or a fragment thereof having the same catalytic activity as the protein it is derived from, which protein or fragment thereof, alone or when provided as molecular complex, referred to as base editing complex herein, has the capacity to mediate a targeted base modification, i.e., the conversion of a base of interest resulting in a point mutation of interest which in turn can result in a targeted mutation, if the base conversion does not cause a silent mutation, but rather a conversion of an amino acid encoded by the codon comprising the position to be converted with the base editor. Usually, base editors are thus used as molecular complex. Base editors, including, for example, CBEs (base editors mediating C to T conversion) and ABEs (adenine base editors mediating A to G conversion), are powerful tools to introduce direct and programmable mutations without the need for double-stranded cleavage (Komor et al., Nature, 2016, 533(7603), 420-424; Gaudelli et al., Nature, 2017, 551, 464-471). In general, base editors are composed of at least one DNA targeting module and a catalytic domain that deaminates cytidine or adenine. All four transitions of DNA (A→T to G→C and C→G to T→A) are possible as long as the base editors can be guided to the target site. Originally developed for working in mammalian cell systems, both BEs and ABEs have been optimized and applied in plant cell systems. Efficient base editing has been shown in multiple plant species (Zong et al., Nature Biotechnology, vol. 25, no. 5, 2017, 438-440; Yan et al., Molecular Plant, vol. 11, 4, 2018, 631-634; Hua et al., Molecular Plant, vol. 11, 4, 2018, 627-630). Base editors have been used to introduce specific, directed substitutions in genomic sequences with known or predicted phenotypic effects in plants and animals. But they have not been used for directed mutagenesis targeting multiple sites within a genetic locus or several loci to identify novel or optimized traits.

As used herein, the terms “(regeneration) booster”, “(regeneration) booster gene”, “(regeneration) booster polypeptide”, “boost polypeptide”, “boost gene” and “boost factor”, refer to a protein/peptide(s), or a (poly)nucleic acid fragment encoding the protein/polypeptide, causing improved plant regeneration of transformed or gene edited plant cells, which may be particularly suitable for improving genome engineering, i.e., the regeneration of a modified plant cell after genome engineering. Such protein/polypeptide may increase the capability or ability of a plant cell, preferably derived from somatic tissue, embryonic tissue, callus tissue or protoplast, to regenerate in an entire plant, preferably a fertile plant. Thereby, they may regulate somatic embryo formation (somatic embryogenesis) and/or they may increase the proliferation rate of plant cells. The regeneration of transformed or gene edited plant cells may include the process of somatic embryogenesis, which is an artificial process in which a plant or embryo is derived from a single somatic cell or group of somatic cells. Somatic embryos are formed from plant cells that are not normally involved in the development of embryos, i.e. plant tissue like buds, leaves, shoots etc. Applications of this process may include: clonal propagation of genetically uniform plant material; elimination of viruses; provision of source tissue for genetic transformation; generation of whole plants from single cells, such as protoplasts; development of synthetic seed technology. Cells derived from competent source tissue may be cultured to form a callus. Further, the term “regeneration booster” may refer to any kind of chemical having a proliferative and/or regenerative effect when applied to a plant cell, tissue, organ, or whole plant in comparison to a no-treated control. The particular artificially created regeneration booster polypeptides according to the present invention may have the dual function of increasing plant regeneration as well as increasing desired genome modification and gene editing outcomes.

A “catalytically active fragment” as used herein means a fragment of an enzyme, particularly of a multi-domain enzyme as Pol ⊖, wherein the catalytically active fragment is still enzymatically active and acts on the same target sequence as the cognate full-length enzyme, wherein the catalytically active fragment may have a substrate specificity and/or catalytic mechanism different to the full-length enzyme in the case of multidomain enzymes (cf. Black et al., 2019, supra, for Pol θ). Individual catalytical domains of an enzyme (e.g., the HNH and the RuvC domain of Cas9) may be presented as individual catalytical fragments. For enzymes exclusively comprising one catalytical domain, for example, a truncated version can serve as catalytically active fragment, wherein the fragment still fulfills the same or comparable function (i.e., catalytic activity) as the full-length or wild-type enzyme.

A “CRISPR nuclease”, as used herein, is a specific form of a site-directed nuclease and refers to any nucleic acid guided nuclease which has been identified in a naturally occurring CRISPR system, which has subsequently been isolated from its natural context, and which preferably has been modified or combined into a recombinant construct of interest to be suitable as tool for targeted genome engineering. Any CRISPR nuclease can be used and optionally reprogrammed or additionally mutated to be suitable for the various embodiments according to the present invention as long as the original wild-type CRISPR nuclease provides for DNA recognition, i.e., binding properties. CRISPR nucleases also comprise mutants or catalytically active fragments or fusions of a naturally occurring CRISPR effector sequences, or the respective sequences encoding the same. A CRISPR nuclease may in particular also refer to a CRISPR nickase or even a nuclease-dead variant of a CRISPR polypeptide having endonucleolytic function in its natural environment. A variety of different CRISPR nucleases/systems and variants thereof are meanwhile known to the skilled person and include, inter alia, CRISPR/Cas systems, including CRISPR/Cas9 systems (EP2771468), CRISPR/Cpf1 systems (EP3009511B11), CRISPR/C2C2 systems, CRISPR/CasX systems, CRISPR/CasY systems, CRISPR/Cmr systems, CRISPR/MAD systems, including, for example, CRISPR/MAD7 systems (WO2018236548A1) and CRISPR/MAD2 systems, CRISPR/CasZ systems and/or any combination, variant, or catalytically active fragment thereof. A nuclease may be a DNAse and/or an RNAse, in particular taking into consideration that certain CRISPR effector nucleases have RNA cleavage activity alone, or in addition to the DNA cleavage activity.

A “CRISPR system” as used herein may be understood as a combination of a CRISPR nuclease or CRISPR effector, or a nickase or a nuclease-dead variant of said nuclease, or a functional active fragment or variant thereof together with the cognate guide RNA (or pegRNAs for a Prime Editing CRISPR system, or a crRNA) guiding the relevant CRISPR nuclease.

A “genome” as used herein is to be understood broadly and comprises any kind of genetic information (RNA/DNA) inside any compartment of a living cell. In the context of a “genome modification”, the term thus also includes artificially introduced genetic material, which may be transcribed and/or translated, inside a living cell, for example, an episomal plasmid or vector, or an artificial DNA integrated into a naturally occurring genome.

The term of “genome engineering” as used herein refers to all strategies and techniques for the genetic modification of any genetic information (DNA and RNA) or genome of a plant cell, comprising genome transformation, genome editing, but also including less site-specific techniques, including TILLING and the like. As such, “genome editing” (GE) more specifically refers to a special technique of genome engineering, wherein a targeted, specific modification of any genetic information or genome of a plant cell. As such, the terms comprise gene editing of regions encoding a gene or protein, but also the editing of regions other than gene encoding regions of a genome. It further comprises the editing or engineering of the nuclear (if present) as well as other genetic information of a plant cell, i.e., of intronic sequences, non-coding RNAs, miRNAs, sequences of regulatory elements like promoter, terminator, transcription activator binding sites, cis or trans acting elements. Furthermore, “genome engineering” also comprises an epigenetic editing or engineering, i.e., the targeted modification of, e.g., DNA methylation or histone modification, such as histone acetylation, histone methylation, histone ubiquitination, histone phosphorylation, histone sumoylation, histone ribosylation or histone citrullination, possibly causing heritable changes in gene expression.

A “genome modification system” as used herein refers to any DNA, RNA and/or amino acid sequence introduced into the cell, on a suitable vector and/or coated on a particles and/or directly introduced. A “genome editing” system more specifically refers to any DNA, RNA and/or amino acid sequence introduced into the cell, on a suitable vector and/or coated on a particles and/or directly introduced, wherein the “genome editing system” comprises at least one component being, encoding, or assisting a site-directed nuclease, nickase or inactivated variant thereof in modifying and/or repairing a genomic target site.

A “genomic target sequence” as used herein refers to any part of the nuclear and/or organellar genome of a plant cell, whether encoding a gene/protein or not, which is the target of a site-directed genome editing or gene editing experiment.

The term “operatively linked”, “operably linked” or “functionally linked” specifically in the context of molecular constructs, for example plasmids or expression vectors, means that one element, for example, a regulatory element, or a first protein-encoding sequence, is linked in such a way with a further part so that the protein-encoding nucleotide sequence, i.e., is positioned in such a way relative to the protein-encoding nucleotide sequence on, for example, a nucleic acid molecule that an expression of the protein-encoding nucleotide sequence under the control of the regulatory element can take place in a living cell.

A “plant material” as used herein refers to any material which can be obtained from a plant during any developmental stage. The plant material can be obtained either in planta or from an in vitro culture of the plant or a plant tissue or organ thereof. The term thus comprises plant cells, tissues and organs as well as developed plant structures as well as sub-cellular components like nucleic acids, polypeptides and all chemical plant substances or metabolites which can be found within a plant cell or compartment and/or which can be produced by the plant, or which can be obtained from an extract of any plant cell, tissue or a plant in any developmental stage. The term also comprises a derivative of the plant material, e.g., a protoplast, derived from at least one plant cell comprised by the plant material. The term therefore also comprises meristematic cells or a meristematic tissue of a plant.

A “PLT” as used herein is means a regeneration booster from the PLETHORA family. Plant development is characterized by repeated initiation of meristems, regions of dividing cells that give rise to new organs. During lateral root (LR) formation, new LR meristems are specified to support the outgrowth of LRs along a new axis. The determination of the sequential events required to form this new growth axis has been hampered by redundant activities of key transcription factors. The effects of three PLETHORA (PLT) transcription factors, PLT3, PLT5, and PLT7, during LR outgrowth were already characterized. It was found that in plt3/plt5/plt7 triple mutants, the morphology of lateral root primordia (LRP), the auxin response gradient, and the expression of meristem/tissue identity markers are impaired from the “symmetry-breaking” periclinal cell divisions during the transition between stage I and stage II, wherein cells first acquire different identities in the proximodistal and radial axes. Particularly, PLT1, PLT2, and PLT4 genes that are typically expressed later than PLT3, PLT5, and PLT7 during LR outgrowth are not induced in the mutant primordia, rendering “PLT-null” LRP. Reintroduction of any PLT clade member in the mutant primordia completely restores layer identities at stage II and rescues mutant defects in meristem and tissue establishment. Therefore, all PLT genes can activate the formative cell divisions that lead to de novo meristem establishment and tissue patterning associated with a new growth axis (Du and Scheres, PNAS 2017, https://doi.org/10.1073/pnas.1714410114). Still, the role of PLT proteins and variants thereof in gene editing and to promote gene editing in a concerted manner together with other regeneration boosters to increase cell regeneration during non-naturally occurring processes like genome editing was not studied in the above cited prior art.

The terms “plant”, “plant organ”, or “plant cell” as used herein refer to a plant organism, a plant organ, differentiated and undifferentiated plant tissues, plant cells, seeds, and derivatives and progeny thereof. Plant cells include without limitation, for example, cells from seeds, from mature and immature embryos, meristematic tissues, seedlings, callus tissues in different differentiation states, leaves, flowers, roots, shoots, male or female gametophytes, sporophytes, pollen, pollen tubes and microspores, protoplasts, macroalgae and microalgae. The different eukaryotic cells, for example, animal cells, fungal cells or plant cells, can have any degree of ploidity, i.e. they may either be haploid, diploid, tetraploid, hexaploid or polyploid.

The term “plant parts” as used herein includes, but is not limited to, isolated and/or pre-treated plant parts, including organs and cells, including protoplasts, callus, leaves, stems, roots, root tips, anthers, pistils, seeds, grains, pericarps, embryos, pollen, sporocytes, ovules, male or female gametes or gametophytes, cotyledon, hypocotyl, spike, floret, awn, lemma, shoot, tissue, petiole, cells, and meristematic cells.

As used herein “a preselected site”, “predetermined site” or “predefined site” indicates a particular nucleotide sequence in the genome (e.g. the nuclear genome, or the organellar genome, including the mitochondrial or chloroplast genome) at which location it is desired to insert, replace and/or delete one or more nucleotides. The predetermined site is thus located in a “genomic target sequence/site” of interest and can be modified in a site-directed manner using a site- or sequence-specific genome editing system.

A “Prime Editing system” as used herein refers to a system as disclosed in Anzalone et al. (2019). Search-and-replace genome editing without double-strand breaks (DSBs) or donor DNA. Nature, 1-1). Base editing as detailed above, does not cut the double-stranded DNA, but instead uses the CRISPR targeting machinery to shuttle an additional enzyme to a desired sequence, where it converts a single nucleotide into another. Many genetic traits in plants and certain susceptibility to diseases caused by plant pathogens are caused by a single nucleotide change, so base editing offers a powerful alternative for GE. But the method has intrinsic limitations, and is said to introduce off-target mutations which are generally not desired for high precision GE. In contrast, Prime Editing (PE) systems steer around the shortcomings of earlier CRISPR based GE techniques by heavily modifying the Cas9 protein and the guide RNA. The altered Cas9 only “nicks” a single strand of the double helix, instead of cutting both. The new guide RNA, called a pegRNA (prime editing extended guide RNA), contains an RNA template for a new DNA sequence, to be added to the genome at the target location. That requires a second protein, attached to Cas9 or a different CRISPR effector nuclease: a reverse transcriptase enzyme, which can make a new DNA strand from the RNA template and insert it at the nicked site. To this end, an additional level of specificity is introduced into the GE system in view of the fact that a further step of target specific nucleic acid::nucleic acid hybridization is required. This may significantly reduce off-target effects. Further, the PE system may significantly increase the targeting range of a respective GE system in view of the fact that BEs cannot cover all intended nucleotide transitions/mutations (C→A, C→G, G→C, G→T, A→C, A→T, T→A, and T→G) due to the very nature of the respective systems, and the transitions as supported by BEs may require DSBs in many cell types and organisms.

As used herein, a “regulatory sequence”, or “regulatory element” refers to nucleotide sequences which are not part of the protein-encoding nucleotide sequence but mediate the expression of the protein-encoding nucleotide sequence. Regulatory elements include, for example, promoters, cis-regulatory elements, enhancers, introns or terminators. Depending on the type of regulatory element it is located on the nucleic acid molecule before (i.e., 5′ of) or after (i.e., 3′ of) the protein-encoding nucleotide sequence. Regulatory elements are functional in a living plant cell.

The terms “transformation”, “transfection”, “transformed”, and “transfected” are used interchangeably herein for any kind of introduction of a material, including a nucleic acid (DNA/RNA), amino acid, chemical, metabolite, nanoparticle, microparticle and the like into at least one cell of interest by any kind of physical (e.g., bombardment), chemical or biological (e.g., Agrobacterium) way of introducing the relevant at least one material. A variety of different techniques is available to the skilled person for various types of target cells, tissues, organs, or materials of interest to be transformed/transfected.

The term “transgenic” as used according to the present disclosure refers to a plant, plant cell, tissue, organ or material which comprises a gene or a genetic construct, comprising a “transgene” that has been transferred into the plant, the plant cell, tissue organ or material by natural means or by means of transformation techniques from another organism. The term “transgene” comprises a nucleic acid sequence, including DNA or RNA, or an amino acid sequence, or a combination or mixture thereof. Therefore, the term “transgene” is not restricted to a sequence commonly identified as “gene”, i.e. a sequence encoding a protein. It can also refer, for example, to a non-protein encoding DNA or RNA sequence, or part of a sequence. Therefore, the term “transgenic” generally implies that the respective nucleic acid or amino acid sequence is not naturally present in the respective target cell, including a plant, plant cell, tissue, organ or material. The terms “transgene” or “transgenic” as used herein thus refer to a nucleic acid sequence or an amino acid sequence that is taken from the genome of one organism, or produced synthetically, and which is then introduced into another organism, in a transient or a stable way, by artificial techniques of molecular biology, genetics and the like.

As used herein, the term “transient” implies that the tools, including all kinds of nucleic acid (RNA and/or DNA) and polypeptide-based molecules optionally including chemical carrier molecules, are only temporarily introduced and/or expressed and afterwards degraded by the cell, whereas “stable” implies that at least one of the tools is integrated into the nuclear and/or organellar genome of the cell to be modified. “Transient expression” refers to the phenomenon where the transferred protein/polypeptide and/or nucleic acid fragment encoding the protein/polypeptide is expressed and/or active transiently in the cells, and turned off and/or degraded shortly with the cell growth. Transient expression thus also implies a stably integrated construct, for example, under the control of an inducible promoter as regulatory element, to regulate expression in a fine-tuned manner by switching expression on or off.

As used herein, “upstream” indicates a location on a nucleic acid molecule which is nearer to the 5′ end of said nucleic acid molecule. Likewise, the term “downstream” refers to a location on a nucleic acid molecule which is nearer to the 3′ end of said nucleic acid molecule. For avoidance of doubt, nucleic acid molecules and their sequences are typically represented in their 5′ to 3′ direction (left to right).

The terms “vector”, or “plasmid (vector)” refer to a construct comprising, inter alia, plasmids or (plasmid) vectors, cosmids, artificial yeast- or bacterial artificial chromosomes (YACs and BACs), phagemides, bacterial phage based vectors, Agrobacterium compatible vectors, an expression cassette, isolated single-stranded or double-stranded nucleic acid sequences, comprising sequences in linear or circular form, or amino acid sequences, viral vectors, viral replicons, including modified viruses, and a combination or a mixture thereof, for introduction or transformation, transfection or transduction into any eukaryotic cell, including a plant, plant cell, tissue, organ or material according to the present disclosure. A “nucleic acid vector, for instance, is a DNA or RNA molecule, which is used to deliver foreign genetic material to a cell, where it can be transcribed and optionally translated. Preferably, the vector is a plasmid comprising multiple cloning sites. The vector may further comprise a “unique cloning site” a cloning site that occurs only once in the vector and allows insertion of DNA sequences, e.g. a nucleic acid cassette or components thereof, by use of specific restriction enzymes. A “flexible insertion site” may be a multiple cloning site, which allows insertion of the components of the nucleic acid cassette according to the invention in an arrangement, which facilitates simultaneous transcription of the components and allows activation of the RNA activation unit.

Whenever the present disclosure relates to the percentage of the homology or identity of nucleic acid (RNA or DNA) or amino acid sequences to each other, this identity implies the identity over the entire length of the sequences to be compared to each other, wherein these identity or homology values define those as obtained by using the EMBOSS Water Pairwise Sequence Alignments (nucleotide) program (http://www.ebi ac.uk/Tools/psa/enboss water/nucleotide.html) nucleic acids or the EMBOSS Water Pairwise Sequence Alignments (protein) program (htp://www.ebi ac.uk/Tools/psa/emboss water/) for amino acid sequences. Those tools provided by the European Molecular Biology Laboratory (EMBL) European Bioinformatics Institute (EBI) for local sequence alignments use a modified Smith-Waterman algorithm (see http://www.ebiac.uk/Tools/psa/and Smith, T. F. & Waterman, M. S. “Identification of common molecular subsequences” Journal of Molecular Biology, 1981 147 (1):195-197). When conducting an alignment, the default parameters defined by the EMBL-EBI are used. Those parameters are (i) for amino acid sequences: Matrix=BLOSUM62, gap open penalty=10 and gap extend penalty=0.5 or (ii) for nucleic acid sequences: Matrix=DNAfull, gap open penalty=10 and gap extend penalty=0.5.

Whenever the present disclosure refers to a POLQ gene, a complete POLQ gene is meant, or a functional domain thereof, wherein such a functional domain may be independently selected from the three domains of Pol ⊖, namely a (i)N-terminal helicase-like domain, a (ii) large central domain harboring a Rad51 interaction motif, and/or a (iii)C-terminal polymerase domain. The same applies, whenever the present disclosure refers to the expression of Pol θ.

This is to be understood as expression of the full length enzyme, or the expression of at least one of the functional domain thereof independently selected from the three domains of Pol ⊖, namely a (i)N-terminal helicase-like domain, a (ii) large central domain harboring a Rad51 interaction motif, and a (iii)C-terminal polymerase domain, wherein the at least one domain can be provided alone, or in combination together with another Pol ⊖, or in combination, e.g., as fusion, with another effector domain, e.g. a CRISPR Cas system, a zinc finger nuclease (ZFN) system, a transcription activator-like nuclease (TALEN) system, or a meganuclease system component, or any combination, variant (for example, nickase, nuclease-dead variant), or catalytically active fragment thereof.

### DETAILED DESCRIPTION

The present invention is based upon the finding that DNA polymerase theta (Pol θ)-originally described as leading enzyme during endogenous MMEJ and HMEJ pathways—is surprisingly significantly improving genome editing (GE) outcomes, preferably in plant cells, when the Pol θ-meditated pathway is activated in a targeted manner, not shut-down or decreased as speculated in previous reports.

In a first aspect of the present invention, there may thus be provided a method for targeted genome modification in a eukaryotic cell, preferably for increasing targeted insertion of a DNA insert at at least one target genomic sequence in a eukaryotic cell, comprising or consisting of the steps: a) providing at least one eukaryotic cell to be modified; b) promoting DNA polymerase theta (Pol θ) activity, or of at least one functional domain thereof, in said cell, wherein the promotion of Pol θ activity increases the DNA insertion efficiency during genome modification; c) introducing into the at least one eukaryotic cell (i) at least one genome modification system, preferably a genome editing system comprising at least one site-directed nuclease, nickase or an inactivated nuclease, preferably a nucleic acid guided nuclease, nickase or an inactivated nuclease, or a sequence encoding the same, and optionally at least one guide molecule, or a sequence encoding the same and (ii) at least one single-stranded or double-stranded DNA insert, or a sequence encoding the same; d) cultivating the at least one eukaryotic cell under conditions allowing (i) the promotion of DNA polymerase Pol θ (Pol θ) activity; and (ii) the activity of the at least one genome editing system and the at least one DNA insert and optionally the at least one guide molecule; and e) obtaining at least one modified cell comprising the sequence of the DNA insert integrated at or close to the target genomic site; and f) optionally: obtaining a eukaryotic organism, plant tissue, organ or seed regenerated from the at least one modified cell.

In the various embodiments as disclosed herein for the above method, steps (b) and (c) may take place simultaneously, or subsequently, for promoting plant cell proliferation and/or to assist in a targeted modification of at least one genomic target sequence of a eukaryotic cell of interest.

A “promotion” of Pol θ activity in the sense of the present invention has to be understood broadly and implies a direct upregulation or overexpression of Pol ⊖, or of a functional domain/fragment thereof, or an indirect promotion of Pol θ activity by influencing the stability or half-half of Pol θ by any chemical or biological agent, or the indirect promotion of Pol θ activity by pathway engineering so that a DNA repair pathway hampering or reducing the activity of Pol θ is modified in a targeted manner to promote Pol θ activity. A promotion or promoting of DNA Pol θ activity may thus imply any upregulation or enhanced activity of the Pol θ activity in comparison to a control cell. This may imply the overexpression of the enzyme to achieve higher levels of enzyme (i.e., more enzyme/cell on a quantitative scale), the upregulation via expression of a heterologous/recombinant Pol θ in a target cell (i.e., a particularly active variant of Pol θ), or a catalytically active fragment thereof in a cell of interest, the pathway engineering, meaning that cellular pathways competing with Pol θ activity are regulated in a way that the Pol θ pathway and thus the Pol θ activity is improved, or any chemical or biological agent that, when provided to an organism or to a cell culture, favors the Pol θ pathway and/or activity.

In one embodiment, it is preferred, if the promotion of Pol θ activity is to be understood as promotion of the activity of the full length Pol θ, or as promotion of at least one functional domain (or, as synonymously used herein, of a functional fragment thereof) of Pol θ. As described above, Pol θ consists of three individual domains, which are an (i)N-terminal helicase-like domain, a (ii) large central domain harboring a Rad51 interaction motif, and a (iii) C-terminal polymerase domain.

It is especially preferred that the promotion of Pol θ activity is achieved via direct upregulation or overexpression of the full length Pol θ, or by direct upregulation or overexpression of at least the (iii)C-terminal polymerase domain of Pol θ as preferred functional domain to be promoted.

Likewise, an indirect promotion can also be achieved by specifically promoting the activity of at least the functional domain of the C-terminal polymerase domain of Pol θ.

In certain embodiments, promoting of Pol θ may be performed by enhancing the cellular level of polymerase Pol θ. Obviously, this can be achieved by overexpression of a recombinant POLQ gene, by activating expression of an endogenous POLQ gene, by activating the pathway leading to Pol θ expression, by providing new variants of Pol θ to a cell of interest (in a stable or in a transient manner) and the like.

Preferred embodiments may include, for example, promoting Pol θ by expressing a heterologous POLQ sequence encoding a heterologous Pol θ and/or modification of the endogenous POLQ sequence to achieve a promotion of Pol θ activity and/or expressing a heterologous POLQ functional domain and/or modification of at least one functional domain of the endogenous POLQ sequence to achieve a promotion of Pol θ activity. In certain preferred embodiments, the Pol θ activity may be promoted indirectly by the inhibition of competing repair pathways (mainly: HR and NHEJ) and, in turn, by favoring the MMEJ/HMEJ pathway.

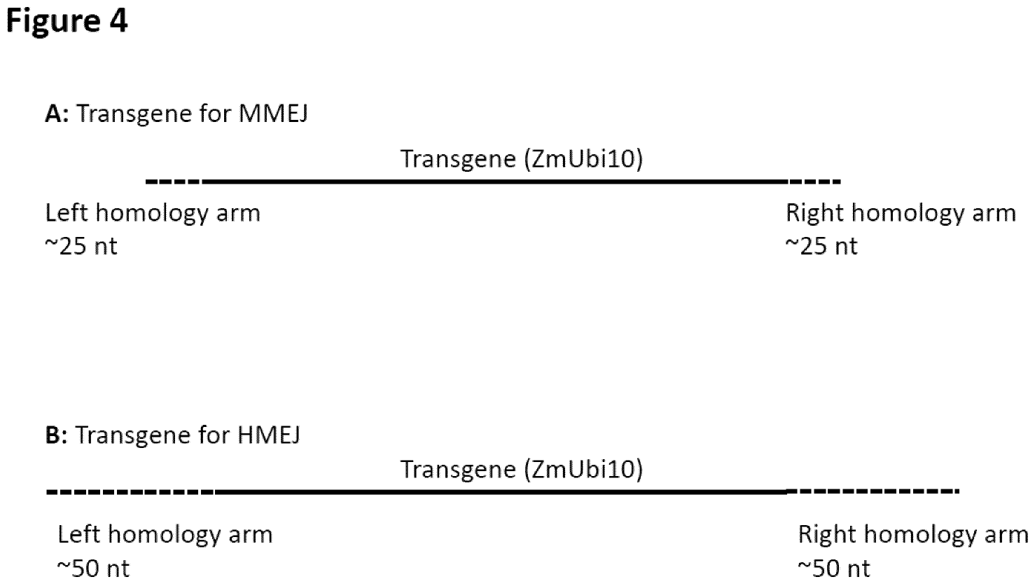

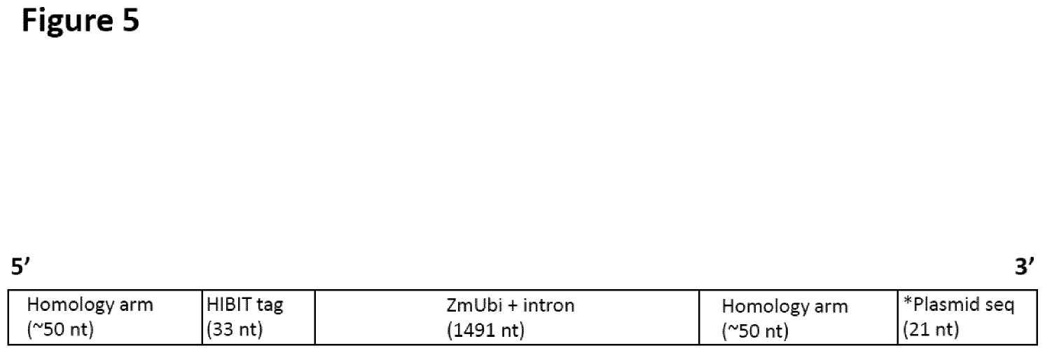

A “DNA insert” as used herein in the context of the methods of the present invention is to be distinguished from a repair template (RT), which understanding is closely related to the naturally occurring Pol θ repair pathway, which is used herein in a modified manner to optimize GE outcomes. The pronounced functional difference is that for HDR, the repair template serves as a template, which means that the DNA strand is synthesized based on the sequence of the RT (recombination), whereas for MMEJ/HMEJ the DNA insert is itself inserted (except for the homology arm (for MMEJ<25 nt and for HMEJ 25-2,000 nt)). Functionally, MMEJ/HMEJ-mediated repair, if used for genome editing, can thus be qualified as an end joining rather than a recombination so that the terms “DNA insert” and “RT” are used differently before this technical background. The methods of the present invention, therefore, exploit the naturally occurring MMEJ/HMEJ pathways to provide improved GE strategies. FIGS. 4 and 5 more specifically illustrate the specific features of a DNA insert (named “Transgene (ZmUbi10)” in FIG. 4) in the sense of the present invention and suitable DNA insert design strategies. In certain embodiments, a DNA insert may also be understood as an RNA insert, or as an insert comprising RNA and DNA portions, as long as this DNA and/or RNA insert is intended to be inserted during MMEJ/HMEJ, or wherein the RNA insert serves as template for the DNA insert.

In one embodiment of the methods as disclosed herein, the DNA polymerase Pol θ sequence, or the POLQ sequence encoding the same, may be selected from the group consisting of SEQ ID NO: 1 to SEQ ID NO: 26, or a sequence, including a functional domain, having at least at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity thereto. Of course, any other naturally occurring Pol θ sequence, or the POLQ sequence encoding the same, can be the subject of a targeted modification, whereas the above sequences may be representative candidates suitable for use in a plant cell. For plants as for other eukaryotic organisms, it may generally be preferably to use a phylogenetically related POLQ sequence for phylogenetically related cells when the activation of Pol θ is achieved via overexpression of a recombinant Pol θ. For example, it will be preferred to use a monocot-derived POLQ sequence for us in the same, or another monocot plant cell.

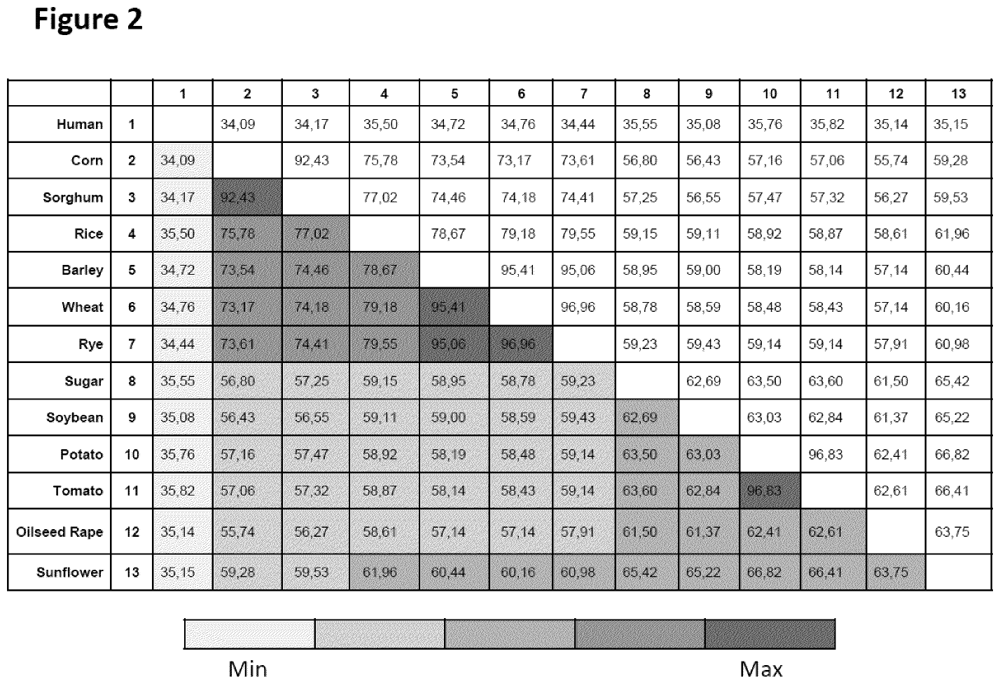

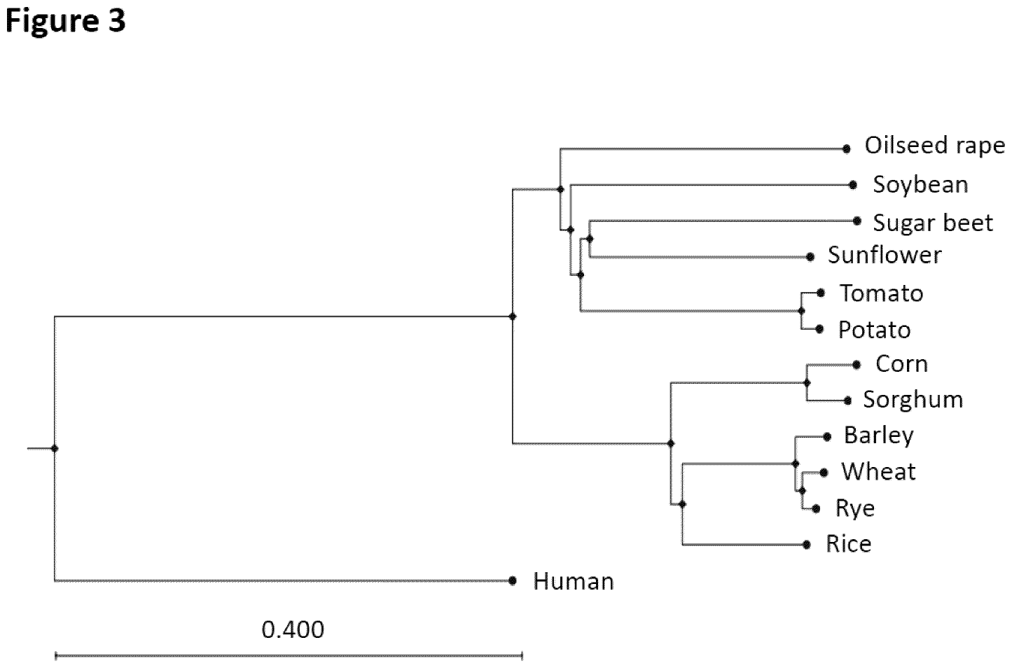

Still, certain POLQ sequences, for example certain Zea mays sequences, may also perform particularly well in a dicot plant like Beta spp., in case the target cell specific regulatory elements are used and a suitable codon optimization for the target cell is performed before to obtain sufficient expression levels of the recombinantly produced enzyme. The nature of the specific Pol theta enzymes as analyzed in detail herein and the relationship between these sequences is illustrated in FIGS. 2 and 3.

Technically, the length of the DNA insert according to the various aspects and methods as disclosed herein can be anything up to 30 kb including homology arms. So the edit could be as short as 1 nt but then to form the complete insert one would need to add the homology arms. As there are no technical restrictions, the DNA insert design and the length will thus be rather flexible depending on the genomic edit to be inserted.

In other embodiment, the DNA insert may be presented as a ribonucleic acid (RNA) molecule, or a molecule comprising at least one RNA portion, as precursor molecule, and this precursor can be additionally provided together with the methods as disclosed herein as DNA insert, or more particularly, as precursor thereof. The RNA-based precursor of interest can be used directly (as RNA) or indirectly via cDNA synthesis. In certain embodiments, at least one repair template (RT), which may be a single-stranded or double-stranded nucleic acid molecule, can be additionally provided together with the methods as disclosed herein. Therefore, the RT may be a single-stranded or double-stranded DNA molecule and/or a single-stranded or double-stranded RNA molecule, or a hybrid between RNA and DNA, wherein the RNA and DNA portion may be covalently or non-covalently linked to each other.

Using RNA as an RT and/or as precursor for a DNA insert can represent an attractive alternative for expanding the flexibility during genome editing, as many copies of the RNA can be produced from a limited number of DNA templates in vivo. This approach can thus remove the need to deliver massive amount of RT to a cell, e.g., via particle bombardment. Further, a frequent problem with single copy of RT delivered via Agrobacterium mediated transformation can be avoided.

All sequence identity ranges provided herein in relation to a parent nucleotide or amino acid sequence as provided in the attached sequence listing take into consideration that a nucleotide sequence representing the coding sequence of a gene may be codon-optimized when used in a different organism. An amino acid sequence representing an enzyme/structural protein may be truncated (to provide only one domain, or a more compact form of a(n) enzyme/protein of interest) or it may contain a mutation at a position not directly influencing the function of the cognate wild-type or reference sequence. Sequence identity ranges are thus disclosed and claimed under the proviso that the such identified nucleotide and/or amino acid sequence still overall fulfills the same function as the respective parent sequence the sequence identity refers to.

In certain embodiments, the activity of DNA polymerase Pol θ may be further promoted by modulating, preferably silencing, components of at least one competing DNA repair pathway, wherein the component belongs to the NHEJ or HDR repair pathway, or the sequence encoding the same, is selected from the group consisting of SEQ ID NO: 27 to SEQ ID NO: 114, or a sequence having at least at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity thereto.

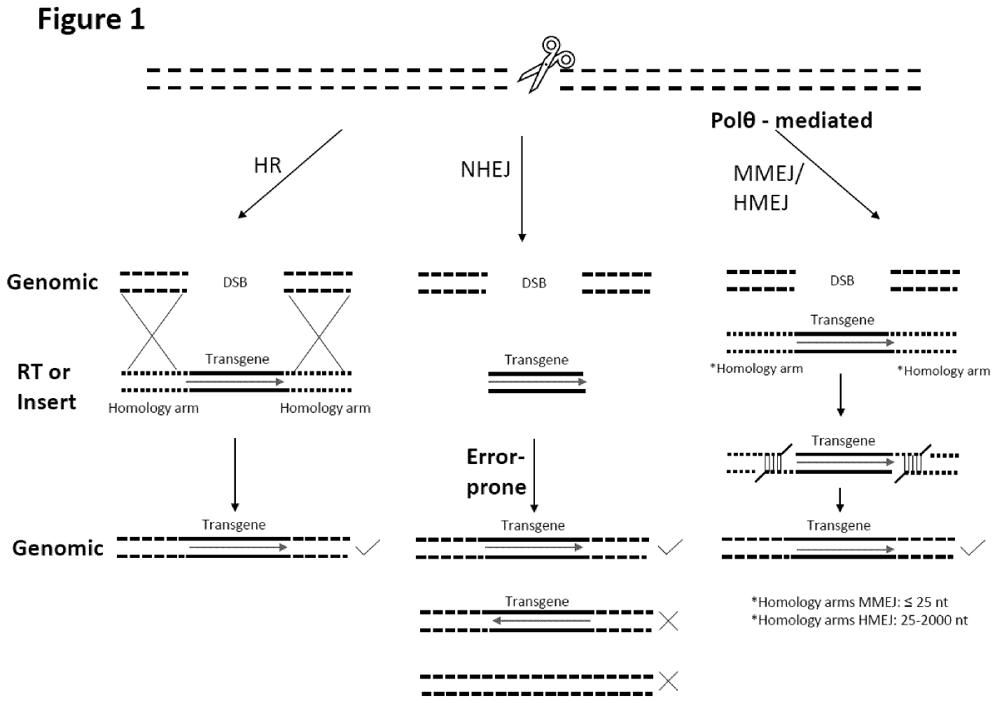

As detailed above, and as illustrated in FIG. 1, several endogenous repair pathways are competing in a eukaryotic cell when it comes up to a repair of a DSB or a single-strand break (SSB). With the knowledge about the impact of Pol θ in the MMEJ/HMEJ pathway and further for improving GE efficiency when Pol θ activity is increased, it was also found that the silencing, down-regulation or decrease of activity of a competing pathway, mainly NHEJ, will shift the endogenous response to the Pol θ-mediated pathway, which is favorable for the methods as disclosed herein. This strategy can be applied together with or alternatively to activating the Pol θ pathway. The present inventor found that particularly the NHEJ-related proteins DNA ligase 4, KU70 and/or KU80 or the HDR-related proteins RPA70C of various plants can be specifically silenced to favor the Pol θ pathway. Whereas a complete knock-out of certain NHEJ or HDR pathway players is conceivable, a silencing or down-regulation may be preferably, which is usually conducted just transiently, as all repair pathways as such are highly conserved and important for cellular genomic integrity so that a stable knock-out instead of a transient knock-down may have certain disadvantages.

In the context of the present disclosure, the terms “silencing” or “RNA interference” or “RNAi” refer to a gene down-regulation mechanism meanwhile demonstrated to exist in all eukaryotes.

The mechanism was first recognized in plants where it was called “post-transcriptional gene silencing” or “PTGS”. In RNAi, small RNAs (of about 21-24 nucleotides) function to guide specific effector proteins (e.g., members of the Argonaute protein family) to a target nucleotide sequence by complementary base pairing. The effector protein complex then down-regulates the expression of the targeted RNA or DNA. Small RNA-directed gene regulation systems were independently discovered (and named) in plants, fungi, worms, flies, and mammalian cells.

Collectively, PTGS, RNA silencing, and co-suppression (in plants); quelling (in fungi and algae); and RNAi (in Caenorhabditis elegans, Drosophila, and mammalian cells) are all examples of small RNA-based gene regulation systems.

In plants, during RNAi mechanism, silencing initiates with the enzyme Dicer and dsRNA is processed to convert the silencing trigger to ˜22-nucleotide, small interfering RNAs (siRNAs).

The antisense strand of siRNA become specific to endonuclease-protein complex, RNA-induced silencing complex (RISC), which then targets the homologous RNA and degrade it at specific site that results in the knock-down of protein expression. RNAi technology may thus be a substitute of complex molecular techniques because of containing several benefits: its specificity and sequence-based gene silencing. Plants can also control viral diseases by RNAi and reveal resistance when having proper anti-sense or hairpin RNAi constructs. In plants, specifically to achieve pathogen resistance, hairpin (hp) dsRNA including small hairpin RNA (shRNA), self-complementary hpRNA, and intron-spliced hpRNA can be formed in vivo using inverse repeat sequences from viral genomes. Among these, PTGS with the highest efficiency was elicited by the method involving self-complementary hairpin RNAs separated by an intron.

High resistance against viruses has been observed in plants even in the presence of inverted repeats of dsRNA-induced PTGS (IR-PTGS). Meanwhile, a variety of different RNAi constructs to be used as silencing construct to be used according to the various aspects and embodiments of the present disclosure are available to the skilled person for plant biotechnology (Younis et al., Int J Biol Sci. 2014; 10(10): 1150-1158). Several methods to induce RNAi, RNAi vectors, in vitro dicing and synthetic molecules are reported.

Mechanistically, introduction of short pieces of double-stranded RNA (dsRNA) and small or short interfering RNA (siRNA) into the cytosol, may initiate the pathway culminating targeted degradation of the specific cellular mRNA, i.e., the target mRNA of the gene transcript to be silenced according to the present invention. Another RNAi molecule are micro RNAs or miRNAs. In spite of similarity in size (20-24 nt), miRNA differ from siRNA in precursor structures, pathway of biogenesis, and modes of action. Artificial miRNAs are known to the skilled person. Both, miRNAs and siRNAs are known to be important regulators of gene expression in plants.

In another embodiment, an RNAi and self-cleaving hammerhead ribozyme may be used to achieve a desired modulation, also on a DNA level (Li Z., Rana T. M. Therapeutic targeting of microRNAs: current status and future challenges. Nat Rev Drug Discov. 2014; 13(8):622-638.).

These reagents allow for targeted control of gene expression by promoting the removal of specific mRNAs from the cytoplasm. The hammerhead ribozyme (HHR), first seen in tobacco ringspot virus satellite RNA, is an example of small nucleolytic RNA molecules capable of self-cleavage (i.e., the name ribozymes). Other autocatalytic (self-cleaving type) small RNA molecules are twister, twister sister, pistol, and hatchet ribozyme. HHRs are composed of a conserved central sequence with three radiating helical domains. Natural HHRs are not true ribozymes as they are only capable of carrying out a single self-cleavage reaction. Synthetic HHRs have been engineered to overcome this by separating the HHR into two components: ribozyme (the part of the HHR which remains unchanged) and substrate (the target sequence that will be cleaved). Another class of suitable modulators for the purpose of the present disclosure are riboswitches. Riboswitches are RNA elements that modulate mRNA expression through binding of a ligand, which is typically a small organic molecule or ion, to its aptamer domain. In one embodiment, the use of a riboswitch might be of interest to modify CPL3 expression in a tightly controlled manner. Meanwhile, a variety of ribozymes and riboswitches types including DNAzymes and temperature-sensitive ribozymes is available to the skilled person (Guha T K, Wai A, Hausner G. Programmable Genome Editing Tools and their Regulation for Efficient Genome Engineering. Comput Struct Biotechnol J. 2017; 15:146-160. Published 2017 Jan 12. doi:10.1016/j.csbj.2016.12.006).

The silencing of the methods as disclosed herein can thus be achieved by a silencing construct being an RNAi silencing construct specifically designed for the transient and preferably activatable down-regulation of at least one pathway protein, or better the sequence encoding the same on RNA level, e.g. the NHEJ pathway, competing with the Pol θ-mediated repair.

The silencing construct may be presented as vector for expression in a cell of interest, or the silencing construct can be prepared ex vivo to be added to a cell, material, tissue, organ or whole organism of interest. In one embodiment, the silencing construct may be operatively linked to a constitutively active promoter. In another embodiment, the silencing construct may be operatively linked to an inducible promoter to control expression of the construct depending on an inducer. Controlled expression of the silencing construct can allow targeted regulation of expression levels of a target protein of interest to be silenced in a temporal (e.g., only during a certain phase of plant development) and/or spatial (e.g., certain plant organs, tissues, cells, or special compartments/organelles) manner. In particular, due to the fact that the target sequences to be silenced play a critical role in plant immunity, it may have significant advantages to restrict silencing in a tempo-spatial and dose dependent way to avoid severe negative effects of the knock-out of plant immunity effectors like CPL proteins due to their highly relevant roles in defence and development.

In a preferred embodiment, a silencing construct of the present invention may be introduced in a transient manner which additionally guarantees that no genetic material is introduced into a plant or plant cell in an inheritable way.

In yet another embodiment, the silencing construct or the RNAi molecule does not share substantial sequence identity with other genomic regions in the genome of the plant cell, tissue, organ, whole plant, or plant material according to the present disclosure is to be understood as a molecule designed in silico based on the information of a sequence to be silenced in combination with the information of the genome to be modified so that the RNAi molecule does not comprise long stretches of identity to other regions in the genome other than the region to be modulated to avoid off-target effects. Usually, the identity to the sequence to be silenced will thus be very high, i.e., at least 90%, 91%, 92%, 93%, 94%, and more preferably at least 95%, 96%, 97%, 98% or even higher than 99%. The substantial identity to other genomic regions in the genome of the plant cell, tissue, organ, whole plant, or plant material will usually be below 25 bp, preferably below 20 bp, 19 bp, 18 bp, 17 bp, 16 bp, more preferably below 15 bp, 14 bp, 13 bp, 12 bp, 11 bp and most preferably below 10 bp of contiguous stretches aligning with another region of a genome of interest.

In yet a further embodiment of the methods as disclosed herein, promotion of the MMEJ/HMEJ pathway is further achieved by co-expressing and/or modulating Pol θ and at least one MRE11 and/or at least one PARP1 protein, wherein a MRE11 and/or PARP1 protein may improve the overall MMEJ/HMEJ pathway, wherein the MRE11 and/or PARP1 protein, or the sequence encoding the same, is selected from the group consisting of SEQ ID NO: 115 to SEQ ID NO: 162 or a sequence having at least at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity thereto.

Particularly MRE11 and/or PARP1 were identified as relevant components of the Pol θ-mediated HMEJ/MMEJ pathway so that Pol θ activity can be indirectly enhanced by providing increased MRE11 and/or PARP1 protein amounts during GE so that these MRE11 and/or PARP1 proteins can enhance efficiency of the Pol θ-mediated HMEJ/MMEJ pathway.

According to the various aspects and embodiments disclosed herein, Pol θ activity will promote targeted genome modification by acting on an endogenous micro-homology mediated end joining (MMEJ) and/or a homology-mediated end-joining (HMEJ) pathway in a favorable manner assisting the targeted GE event to be inserted into a genomic target site of interest.

As central component, the methods of the present invention rely on a site-specific genome/gene editing system, i.e., a rare-cleaving and programmable site-directed nuclease. In preferred embodiments, the at least one site-directed nuclease, nickase or inactivated nuclease, or a sequence encoding the same, may be selected from the group consisting of a CRISPR/Cas system, preferably from a CRISPR/Cas12a or a CRISPR/Cas12b system, including a CRISPR/MAD7 system, a CRISPR/Cfp1 system, or a CRISPR/MAD2 system, from a CRISPR/Cas9 system, a CRISPR/CasX system, a CRISPR/CasY system, a CRISPR/Cas13 system, or a CRISPR/Csm system, a zinc finger nuclease (ZFN) system, a transcription activator-like nuclease (TALEN) system, or a meganuclease system, or any combination, variant, or catalytically active fragment thereof. CRISPR nucleases and systems suitable for the methods as disclosed herein are defined and described above. In other preferred embodiments, the present invention relates to the use of nuclease—Pol θ fusions, wherein Pol θ, or at least one functional domain thereof is fused to at least one site-directed nuclease, nickase or inactivated nuclease, or a sequence encoding the same, may be selected from the group consisting of a Cas protein, preferably from a Cas12a or a Cas12b protein, including a MAD7 protein, a Cfp1 protein, or a MAD2 protein, from a Cas9 protein, a CasX protein, a CasY protein, a Cas13 protein, or a Csm protein, a zinc finger nuclease (ZFN), a transcription activator-like nuclease (TALEN), or a meganuclease, or any combination, variant, or catalytically active fragment thereof.

These nucleases, in comparison to TALEN or ZFN systems, may have the advantage that the RNA-guided CRISPR nuclease, nickase or variant can be easily programmed or re-programmed by just exchange the at least one guiding RNA to a new genomic target site of interest without the need to modify the CRISPR nuclease, nickase or variant as such.

In certain embodiments, CRISPR systems relying on double-strand break inducing nuclease may be used, wherein in other embodiments, a CRISPR system using a nickase or a nuclease-dead variant may be used, for example, a base editor system and/or a prime editing system as defined above.

In certain embodiments, the eukaryotic cell to be modified in a targeted manner with the methods of the present invention will be a plant cell, wherein the method may additionally comprise a step g) of screening for at least one plant tissue, organ, plant or seed regenerated from the at least or modified cell in the TO and/or T1 generation carrying the DNA insert.

In another embodiment, the at least one eukaryotic cell may be selected from a plant cell, or a mammalian cell. In view of the fact that the central Pol θ pathway to be activated along with site directed GE is conserved and present in plant and mammalian cells, including human cells, the methods as disclosed herein are not only suitable for plant genome engineering, but may likewise be employed for GE in mammalian cells, tissues, organs or materials, for example, for therapeutic purposes.

In preferred embodiments of the present disclosure, the at least one eukaryotic cell may be a plant cell, preferably wherein the plant cell may be a plant cell selected from the group consisting of Hordeum, Sorghum, Saccharum, Zea, Setaria, Oryza, Triticum, Secale, Triticale, Malus, Brachypodium, Aegilops, Daucus, Beta, Eucalyptus, Nicotiana, Solanum, Coffea, Vitis, Erythrante, Genlisea, Cucumis, Marus, Arabidopsis, Crucihimalaya, Cardamine, Lepidium, Capsella, Olmarabidopsis, Arabis, Brassica, Eruca, Raphanus, Citrus, Jatropha, Populus, Medicago, Cicer, Cajanus, Phaseolus, Glycine, Gossypium, Astragalus, Lotus, Torenia, Allium, Spinacia or Helianthus, preferably, the plant or plant cell originates from a species selected from the group consisting of Hordeum vulgare, Hordeum bulbusom, Sorghum bicolor, Saccharum officinarium, Zea spp., including Zea mays, Setaria italica, Oryza minuta, Oryza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta spp., including Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Marus notabilis, Arabidopsis arenosa, Arabidopsis lyrata, Arabidopsis thaliana, Crucihimalaya himalaica, Crucihimalaya wallichii, Cardamine nexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Allium tuberosum, Helianthus annuus, Helianthus tuberosus and/or Spinacia oleracea.

In certain embodiments, the method of the present invention may additionally comprise the step of introducing or applying at least one regeneration booster, or a sequence encoding the same, or a regeneration booster chemical.

According to the various aspects and embodiments disclosed herein, it may be suitable to combine genome editing (GE) with the provision of additional regeneration boosting genes/proteins to reduce the cell stress associated with the introduction of the GE tools. Suitable “regeneration boosters” as used herein may be selected based on their functions involved in promoting cell division and plant morphogenesis. In particular, a booster or booster system of interest should be compatible with a given plant without having adverse effects on plant development. The latter point is caused by the fact that naturally occurring booster proteins are usually transcription factors guiding the progression of cell differentiation at different positions in a precise manner and thus have central roles in plant development.

Certain regeneration booster sequences, usually representing transcription factors active during various stages of plant development and also known as morphogenic regulators in plants, are known for long, including the Wuschel (WUS) and babyboom (BBM) class of boosters (Mayer, K. F. et al. Role of WUSCHEL in regulating stem cell fate in the Arabidopsis shoot meristem. Cell 95, 805-815 (1998); Yadav, R. K. et al. WUSCHEL protein movement mediates stem cell homeostasis in the Arabidopsis shoot apex. Genes Dev 25, 2025-2030 (2011); Laux, T., Mayer, K. F., Berger, J. & JQrgens, G. The WUSCHEL gene is required for shoot and floral meristem integrity in Arabidopsis. Development 122, 87-96 (1996); Leibfried, A. et al. WUSCHEL controls meristem function by direct regulation of cytokinin-inducible response regulators. Nature 438, 1172-1175 (2005); for BBM: Hofmann, A Breakthrough in Monocot Transformation Methods, The Plant Cell, Vol. 28: 1989, September 2016). Others, including the RKD (including TaRKD4 disclosed herein as SEQ ID NOs: 52 and 53, or a variant, or a codon-optimized version thereof) and LEC family of transcription factors have been steadily emerging and are meanwhile known to the skilled person (Hofmann, A Breakthrough in Monocot Transformation Methods The Plant Cell, Vol. 28: 1989, September 2016; New Insights into Somatic Embryogenesis: LEAFY COTYLEDON1, BABY BOOM1 and WUSCHEL-RELATED HOMEOBOX4 Are Epigenetically Regulated in Coffea canephora, PLos one August 2013, vol. 8(8), e72160; LEAFY COTYLEDON1-CASEIN KINASE I-TCP15-PHYTOCHROME INTERACTING FACTOR4 Network Regulates Somatic Embryogenesis by Regulating Auxin Homeostasis Plant Physiology_, December 2015, Vol. 169, pp. 2805-2821; A. Cagliari et al. New insights on the evolution of Leafy cotyledon1 (LEC1) type genes in vascular plants Genomics 103 (2014) 380-387, U.S. Pat. No. 6,825,397B1; U.S. Pat. No. 7,960,612B2, WO2016146552A1). The Growth-Regulating Factor (GRF) family of transcription factors, which is specific to plants, is also known to the skilled person. At least nine GRF polypeptides have been identified in Arabidopsis thaliana (Kim et al. (2003) Plant J 36: 94-104), and at least twelve in Oryza sativa (Choi et al. (2004) Plant Cell Physiol 45(7): 897-904). The GRF polypeptides are characterized by the presence in their N-terminal half of at least two highly conserved domains, named after the most conserved amino acids within each domain: (i) a QLQ domain (InterPro accession IPR014978, PFAM accession PF08880), where the most conserved amino acids of the domain are Gln-Leu-Gln; and (ii) a WRC domain (InterPro accession IPR014977, PFAM accession PF08879), where the most conserved amino acids of the domain are Trp-Arg-Cys. The WRC domain further contains two distinctive structural features, namely, the WRC domain is enriched in basic amino acids Lys and Arg, and further comprises three Cys and one His residues in a conserved spacing (CX9CX10CX2H), designated as the Effector of transcription (ET) domain (Ellerstrom et al. (2005) Plant Molec Biol 59: 663-681). The conserved spacing of cysteine and histidine residues in the ET domain is reminiscent of zinc finger (zinc-binding) proteins. In addition, a nuclear localisation signal (NLS) is usually comprised in the GRF polypeptide sequences.

Another class of potential regeneration boosters, yet not studied in detail for their function in artificial genome/gene editing, is the class of PLETHORS (PLT) transcription factors as introduced and defined above.

Regeneration boosters derived from naturally occurring transcription factors, as, for example, BBM or WUS, and variants thereof, may have the significant disadvantage that uncontrolled activity in a plant cell over a certain period of time will have deleterious effects on a plant cell. Therefore, the present inventors conducted a series of in silico work to create fully artificial regeneration booster proteins after a series of multiple sequence alignment, domain shuffling, truncations and codon optimization for various target plants. By focusing on core consensus motifs, it was an object to identify new variants of regeneration boosters not occurring in nature that are particularly suitable for us in methods for genome modifications and gene editing. Various gymnosperm sequences occurring in different species presently not considered as having a regeneration booster activity of described booster genes and proteins were particularly considered in the design process of the new booster sequences.

Based on this work, the present applicant found that specific regeneration boosters, as well as certain modified regeneration boosters naturally acting as transcription factors artificially created (RBPs) perform particularly well in combination with the methods disclosed herein, as they promote regeneration and additionally have the capacity to improve genome modification or gene editing efficiencies. Further, the artificially created and then stepwise selected and tested regeneration boosters do not show pleiotropic effects and are particularly suitable to be used during any kind of genome modification such as gene editing.

In one embodiment, the regeneration booster may comprises at least one of an RBP and/or at least one PLT, preferably wherein the regeneration booster comprises at least one of an RBP, wherein the at least one regeneration booster sequence is individually selected from any one of SEQ ID NOs: 171, 195 to 201, and 209 to 211 or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or an active fragment thereof, or wherein the at least one regeneration booster sequence is encoded by a sequence individually selected from any one of SEQ ID NO: 172, 202 to 208, and 212 to 214 or a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity thereto.

In another embodiment, alone or in combination with using at least one RBP and/or at least one PLT as booster, a conventional booster may be applied so that the regeneration booster may be selected from the group consisting of BBM, WUS, WOX, RKD4, RKD2, including a RKD from Triticum aestivum, GRF (a growth-regulating factor described in the art for its activity as regeneration booster), LEC, or a variant thereof, or a sequence encoding the same.

In one embodiment, a gene encoding a regeneration booster as disclosed herein may comprise at least one regulatory element as detailed below. In view of the fact that the RBP regeneration booster genes disclosed herein are fully artificial, there is no classical “natural” regulatory element, e.g., a promoter, to be used. Therefore, the choice of at least one suitable regulatory element will be guided by the question of the host cell of interest and/or spatio-temporal expression patterns of interest, so that the optimum regulatory elements can be chosen to achieve a specific expression of the at least one regeneration booster gene of interest.

In one embodiment, wherein more than one regeneration booster gene are used, different promoters may be chosen, for example, the promoters having different activities so that the at least two genes can be expressed in a defined and controllable manner to have a stronger expression of a first regeneration booster protein/polypeptide (RBP) and a weaker expression of a second RBP, where a differential expression pattern may be desired.

In one embodiment, at least one regeneration booster, or a sequence encoding the same, or a regeneration booster chemical, can be provided before, simultaneously, or subsequently with/to other tools to be inserted, namely the at least one genome modification system, preferably the genome editing system, and the at least one DNA insert to reduce the number of transformation/transfection acts potentially stressful for a cell. For certain cells sensitive to transformation/transfection, regeneration booster chemicals may thus represent a suitable option, which may be provided before, simultaneously with, or soon after transforming/transfecting further genome or gene editing tools to reduce the cellular stress and to increase transformation and/or editing efficiency by stabilizing a cell and thus by reducing potentially harmful cellular stress responses.

In another embodiment, the genome editing system and the at least one regeneration booster, or the sequence encoding the same, may be provided subsequently or sequentially. By separating the introduction steps, the editing construct DNA integration of the site-directed nuclease, nickase or an inactivated nuclease encoding sequence can be avoided, where transient outcomes are of interest.

In certain embodiments, it is favourable that the at least one regeneration booster is active in a cell before further tools, i.e., the genome modification system and the DNA insert, are introduced to put the cell into a state of low cellular stress before performing genome or gene editing.

In certain embodiments of the methods disclosed herein, at least one first RBP or PLT, or a sequence encoding the same, preferably at least one RBP, most preferably RBP2, or the sequence encoding the same, may be provided and at least one further regeneration booster may be provided selected from: (i) at least one further RBP and/or PLT, or the sequence encoding the same, or a variant thereof, (ii) at least one BBM, or the sequence encoding the same, or a variant thereof, (iii) at least one WOX, including WUS1, WUS2, or WOX5, or the sequence encoding the same, or a variant thereof, (iv) at least one RKD4 or RKD2, including wheat RKD4, or the sequence encoding the same, or a variant thereof, (v) at least one GRF (growth-regulating factor), including Zea mays GRF5 and Zea mays GRF1/TOW, or the sequence encoding the same, or a variant thereof, and/or (vi) at least one LEC sequence, including LEC1 and LEC2, or the sequence encoding the same, or a variant thereof, and wherein the at least one second regeneration booster, or a sequence encoding the same, may be different to the first regeneration booster.

In one embodiment, at least one regeneration booster, or a sequence encoding the same, or a regeneration booster chemical, can be provided simultaneously with other tools to be inserted, namely the at least one genome modification system, preferably the genome editing system to reduce the number of transformation/transfection acts potentially stressful for a cell. For certain cells sensitive to transformation/transfection, regeneration booster chemicals may thus represent a suitable option, which may be provided before, simultaneously with, or soon after transforming/transfecting further genome or gene editing tools to reduce the cellular stress and to increase transformation and/or editing efficiency by stabilizing a cell and thus by reducing potentially harmful cellular stress responses.

In preferred embodiments, in case regeneration booster is provide as expression construct to a cell of interest together with the methods as disclosed herein, the expression construct(s) or plasmid(s), preferably including one encoding RBP2, may be provided to at least one plant cell simultaneously with other tools to decrease the total amount of transformation/transfection steps. However, in the case of a chemical booster, it could be delivered few hours before the other editing components, for example, to the culture medium to prepare the at least one cell for a subsequent transformation/transfection.

For any simultaneous or subsequent introduction of at least one regeneration booster, the regeneration booster and the optional further genome modification or genome editing system should be active, i.e., present in the active protein and/or RNA stage, in one and the same cell to be modified, preferably in the nucleus of the cell, or in an organelle comprising genomic DNA to be modified.

According to the various embodiments and aspects disclosed herein, it may be preferable to use a naturally occurring regeneration booster in addition to an artificial RBP according to the present invention, wherein the naturally occurring regeneration booster, e.g., BBM, WUS½, LEC½, GRF, or a PLT may be derived from a target plant to be transformed, or from a closely related species. For monocot plant modifications, for example, a booster protein with monocot origin (e.g., from Zea mays (Zm)) may be preferred, whereas for dicot plant modifications, a booster protein with dicot origin (e.g., originating from Arabidopsis thaliana (At), or Brassica napus (Bn)) may be preferred. The relevant booster sequences can be easily identified by sequence searches within the published genome data. Notably, regeneration boosters from one plant species may show a certain cross-species applicability so that, for example, a wheat-derived booster gene may be used in Zea mays, and vice versa, or a Arabidopsis—or Brachypodium-derived booster gene may be used in Helianthus, and vice versa. A PLT, WUS, WOX, BBM, LEC, RKD4, or GRF sequence as used herein, or a protein with a comparable regeneration booster function, may thus be derived from any plant species harbouring a corresponding gene encoding the respective booster in its genome.

In a further aspect according the present invention, compatible with all embodiments according to the first aspect as detailed above, there is provided a method for genome modification in a eukaryotic cell, preferably for increasing insertion of a DNA insert at at least one genomic sequence in a eukaryotic cell, wherein the method may comprise or consist of the steps: a) providing at least one eukaryotic cell to be modified; and b) promoting DNA polymerase Pol θ activity in said cell, wherein the method is suitable for random integration of the at least one DNA insert.

In yet a further aspect, there may be provided a genetically modified cell, tissue, organ or material, including a seed, obtainable by a method according to any one of the methods as disclosed herein.

In one embodiment, the cell, tissue, organ or material, including a seed, may be a plant cell, tissue, organ or material, including a seed.

In yet a further aspect, there may be provided a generally applicable expression construct assembly, which may be used according to the methods disclosed herein, wherein the expression construct assembly may comprise (i) at least one vector encoding at least one Pol θ sequence as defined above, and optionally comprising a construct encoding silencing components of at least one MMEJ/HMEJ competing repair pathway as defined above, and optionally a protein aiding Pol θ activity as defined above; and (ii) at least one vector encoding a gene encoding at least one genome editing system, preferably wherein the genome editing system is as defined as above, optionally comprising at least one vector encoding at least one guide molecule as defined above in the context of a CRISPR nuclease or system; and (iii) at least one vector encoding at least one DNA insert, or at least one single-stranded or double-stranded DNA insert as defined for the first aspect above; and (iv) optionally: at least one vector encoding at least one regeneration booster, preferably wherein the regeneration booster is as defined above; wherein (i), (ii), (iii), and/or (iv) may be encoded on the same, or on different vectors.

In one embodiment, the expression construct assembly may further comprise a vector encoding at least one marker, preferably wherein the marker is introduced in a transient manner, see, for example, SEQ ID NO: 165.

In yet a further embodiment, the expression construct assembly may comprise at least one construct or sequence individually selected from any one of SEQ ID NOs: 163 to 165, 170, 173 to 175, and 179 to 183, 185, 187 and 189, or a variant thereof with minor differences not effecting the encoded regions.

In one embodiment, the expression construct assembly may comprise or encode at least one regulatory sequence, wherein the regulatory sequence is selected from the group consisting of a core promoter sequence, a proximal promoter sequence, a cis regulatory sequence, a trans regulatory sequence, a locus control sequence, an insulator sequence, a silencer sequence, an enhancer sequence, a terminator sequence, an intron sequence, and/or any combination thereof.

Notably different components of a genome modification or editing system and/or a regeneration booster sequence and/or a guide molecule and/or a DNA template present on the same vector of an expression vector assembly may be comprise or encode more than one regulatory sequence individually controlling transcription and/or translation.

In one embodiment of the expression construct assembly described above, the construct comprises or encodes at least one regulatory sequence, wherein the regulatory sequence is selected from the group consisting of a core promoter sequence, a proximal promoter sequence, a cis regulatory sequence, a trans regulatory sequence, a locus control sequence, an insulator sequence, a silencer sequence, an enhancer sequence, a terminator sequence, an intron sequence, and/or any combination thereof.

In another embodiment of the expression construct assembly described above, the regulatory sequence comprises or encodes at least one promoter selected from the group consisting of ZmUbi1, BdUbi10, ZmEf1, a double 35S promoter, a rice U6 (OsU6) promoter, a rice actin promoter, a maize U6 promoter, PcUbi4, Nos promoter, AtUbi10, BdEF1, MeEF1, HSP70, EsEF1, MdHMGR1, or a combination thereof.

In a further embodiment of the expression construct assembly described above, the at least one intron is selected from the group consisting of a ZmUbi1 intron, an FL intron, a BdUbi10 intron, a ZmEf1 intron, a AdH1 intron, a BdEF1 intron, a MeEF1 intron, an EsEF1 intron, and a HSP70 intron.

In one embodiment of the expression construct assembly according to any of the embodiments described above, the construct comprises or encodes a combination of a ZmUbi1 promoter and a ZmUbi1 intron, a ZmUbi1 promoter and FL intron, a BdUbi10 promoter and a BdUbi10 intron, a ZmEf1 promoter and a ZmEf1 intron, a double 35S promoter and a AdH1 intron, or a double 35S promoter and a ZmUbi1 intron, a BdEF1 promoter and BdEF1 intron, a MeEF1 promoter and a MeEF1 intron, a HSP70 promoter and a HSP70 intron, or of an EsEF1 promoter and an EsEF1 intron.

In addition, the expression construct assembly may comprise at least one terminator, which mediates transcriptional termination at the end of the expression construct or the components thereof and release of the transcript from the transcriptional complex.

In one embodiment of the expression construct assembly according to any of the embodiments described above, the regulatory sequence may comprise or encode at least one terminator selected from the group consisting of nosT, a double 35S terminator, a ZmEf1 terminator, an AtSac66 terminator, an octopine synthase (ocs) terminator, or a pAG7 terminator, or a combination thereof. A variety of further suitable promoter and/or terminator sequences for use in expression constructs for different plant cells are well known to the skilled person in the relevant field.

A variety of suitable fluorescent marker proteins and fluorophores applicable over the whole spectrum, i.e., for all fluorescent channels of interest, for use in plant biotechnology for visualization of metabolites in different compartments are available to the skilled person, which may be used according to the present invention. Examples are GFP from Aequoria victoria, fluorescent proteins from Anguillajaponica, or a mutant or derivative thereof), a red fluorescent protein, a yellow fluorescent protein, a yellow-green fluorescent protein (e.g., mNeonGreen derived from a tetrameric fluorescent protein from the cephalochordate Branchiostoma lanceolatum), an orange, a red or far-red fluorescent protein (e.g., tdTomato (tdT), or DsRed), and any of a variety of fluorescent and coloured proteins may be used depending on the target tissue or cell, or a compartment thereof, to be excited and/or visualized at a desired wavelength.

All elements of the expression vector assembly can be individually combined. Further, the elements can be expressed in a stable or transient manner, wherein a transient introduction may be preferably. In certain embodiments, individual elements may not be provided as part of a yet to be expressed (transcribed and/or translated) expression vector, but they may be directly transfected in the active state, simultaneously or subsequently, and can form the expression vector assembly within one and the same IIM cell of interest to be modified. For example, it may be reasonable to first transform part of the expression vector assembly encoding a site-directed nuclease, which takes some time until the construct is expressed, wherein the cognate guide molecule is then transfected in its active RNA stage and/or at least one repair template is then transfected in its active DNA stage in a separate and subsequent introduction step to be rapidly available. The at least one regeneration booster sequence and/or the at least one genome modification or editing system and/or the at least one marker may also be transformed as part of one vector, as part of different vectors, simultaneously, or subsequently. The use of too many individual introduction steps should be avoided, and several components can be combined in one vector of the expression vector assembly, to reduce cellular stress during transformation/transfection. In certain embodiments, the individual provision of elements of the at least one regeneration booster sequence and/or the at least one genome modification or editing system and/or the at least one marker and/or the at least one guide molecule and/or the at least one repair template on several vectors and in several introduction steps may be preferable in case of complex modifications relying on all elements so that all elements are functionally expressed and/or present in a cell to be active in a concerted manner.

The present invention is further illustrated by the following non-limiting Examples.

### EXAMPLES

### Example 1: DNA Insert Design—Transgene Properties

Double-stranded oligonucleotides were used as DNA inserts. The structure of the 1.6 kb DNA insert (SEQ ID NO: 163) to target the Zea mays CWI2 gene promoter is visualized in FIGS. 4 and 5. The 1.5 kb sequence (SEQ ID NO: 164) to be integrated was framed with 50 nt homology arms on both sides.

### Example 2: Transformation of Gene Editing Components in Maize Immature Embryo—Delivery Methods

Plasmid Constructs Expressing:

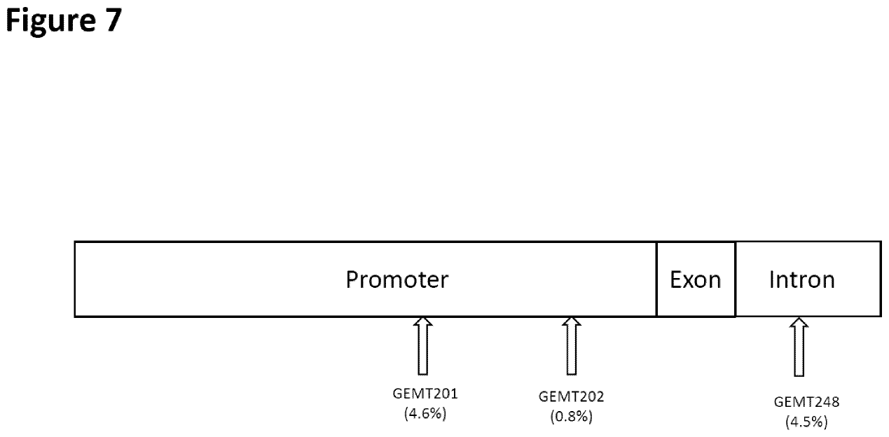

MAD7 (for example, plasmid SEQ ID NO: 165, MAD7 SEQ ID NO: 166 (CDS) and 167 (PRT)) endonuclease and tDTomato (SEQ ID NO: 168 (CDS) and 169 (PRT)), a regeneration booster protein (for example, plasmid SEQ ID NO: 170, RBP2 CDS: 171, RBP2 Protein SEQ ID NO: 172), two guide RNAs (either GEMT201 (SEQ ID Nos: 173 and 176) with GEMT202 (SEQ ID NO: 174 and 177) or GEMT201 with GEMT248 (SEQ ID Nos: 175 and 178)), that direct the endonuclease to the target site (e.g. the CWI2 promoter, see FIGS. 6 and 7) and the double-stranded DNA insert (SEQ ID NO: 179) were co-bombarded into maize immature embryos (genotype A188) using biolistic delivery.

Step 1: Ear Sterilization

Maize ears with immature embryos size 0.5 to 2.5 mm were first sterilized with 10% bleach (8.25% sodium hypochlorite) plus 0.1% Tween 20 for 10 to 20 minutes or 70% ethanol for 10-15 minutes and then washed four times with sterilized H₂O. Sterilized ears were dried briefly in a sterile hood for 5 to 10 minutes.

Step 2: Immature Embryos Isolation for Gold Particle Bombardment

Immature embryos (preferably 1.2-1.5 mm of size, 0.8-1.8 mm also possible) were isolated under sterile conditions by first removing the top third of the kernels from the ears with a sharp scalpel. Then immature embryos were carefully pulled out of the kernel with a spatula. The freshly isolated embryos were placed onto the bombardment target area in an osmotic medium plate (N60SM-no2,4-D medium) with scutellum-side up. Plates were sealed and incubated at 25° C. in darkness for 4-20 hours (preferably 4 hours) before bombardment.

Step 3: Bombardment

First, gold particles were prepared as follows:
1. The stock solution for gold particles can be prepared in advance, at least 1 day prior to bombardment and stored at −20° C. for at least 6 months.
2. Weigh out 10 mg of gold particles (0.4-0.6 μm) into a 1.7 ml centrifuge tube (low retention).
3. Add 1 mL of 100% ethanol (molecular biology grade), and vortex-mix for 2 min and sonicate in an ultrasonic water bath for 15 sec.
4. Pellet the gold particles by centrifuging the tube for 1 min at 3000 rpm in a bench top microcentrifuge and then discard the supernatant.
5. Repeat step 3-4 once.
6. Add 1 ml of 70% ethanol and vortex-mix for 2 min.
7. Incubate the tube for 15 min at room temperature. Mix the contents of the tube about three times during the incubation.
8. Centrifuge the tube at 3000 rpm for 1 min, and then discard the supernatant.
9. Repeat step 6-8 one more time.
10. Add 1 ml of sterile Milli Q water and mix for 2 min or until the particles are completely suspended.
11. Allow the particles to settle down for 1 min at room temperature and then centrifuge the tube at 3000 rpm for 1 min; discard the supernatant.
12. Repeat step 10-11 two more times.
13. Add 1.0 ml of sterile 50% (v/v) glycerol to the gold particles at a final concentration of 10 mg/ml.
14. Store the gold particles at −20° C. until ready to use.

Then, DNA was coated onto the gold particles (for 10 bombardments) as follows:
1. Vortex the previously prepared gold particles that were stored at −20° C. until they are completely re-suspended.
2. Pipet out 100 μl of the gold particle suspension (1 mg) into a 1.7 ml microcentrifuge tube (low retention).
3. Sonicate for 15 sec.
4. While vortex (at low to middle speed), add the following in order to each 100 μl of gold particles in 50% glycerol: 10 μl of DNA 100 μl of 2.5 M CaCl2 (pre-cold on ice) 40 μl of 0.1 M cold spermidine (prepare right before use). (Important: The order of adding gold particles, DNA, CaCl2, and spermidine is important for the proper coating of the gold particles. Spermidine must be prepared fresh and kept on ice).
5. Close the lids and continue vortexing for 5-10 minutes at RT.
6. Allow the DNA-coated gold particles to settle 1 minute, spin for 5 seconds at the top speed, and then remove supernatant.
7. Wash the pellet in 500 μl of 100% Ethanol without disturbing pellet for 1 minute.
8. Remove supernatant without disturbing pellet.
9. Repeat steps 7 and 8 one more time.
10. Finally resuspend the DNA coated gold particles in 120 μl of 100% EtOH (for 10 bombardments). Gently vortex to resuspend.
11. Quickly pipet 10 μl of the well-suspended gold particles with a wide open 20 μl tip from the tube onto the center of a macrocarrier and spread out the gold particles around the macrocarrier evenly (note: the particles tend to form clumps at this point). Air dry and use for bombardment as soon as possible (the DNA-coated gold particles must be used within 2 h).

In a next step, the prepared gold particles were bombarded into the prepared immature embryos (osmotic treatment 4-20 h pre-bombardment by incubation on N60SM-no2,4-D medium) using the following conditions: 3 shots per plate, 100 μg of gold particles per shot, 100 ng—200 ng of each plasmid DNA, and 500 ng of DNA insert per shot and 450-650 psi for 0.6 μm gold particles, at least 650 psi for 0.4 μm gold particles.

After the bombardment, immature embryos were incubated for 16 to 20 h on N60SM-no2,4-D media plates for a second osmotic treatment.

Step 3: Post Bombardment Culture and Regeneration

First, the formation of Type II calli was induced 16-20 h post bombardment. Therefore, embryos with dense fluorescent signals under a fluorescence microscope were selected and transferred from the N60SM-no2,4-D onto a N6-5Ag plate (˜15 embryos per plate) with scutellum-face-up. The embryos on the N6-5Ag plate were incubated at 27° C. in darkness for 14-16 days to induce type II calli. For type II callus regeneration, calli from the bombarded region of the plate were transferred to MRM1 medium and cultured on MRM1 medium at 25° C. in darkness until the somatic embryo matured (˜2 weeks). The mature somatic embryos were then transferred onto MS0 medium in a phytotray for embryo germination. Therefore, they were cultured in the full light chamber at 25° C. until the plants are ready for moving to the greenhouse (˜1 week).

Media Used: 1. N6-5Ag

| Compound | Amount [mg/L] |
|---|---|
| N6 salt | |
| N6 vitamin | |
| 2,4-D | 1.0 |
| Casein | 100.0 |
| Proline | 2,900 |
| Sucrose | 20,000 |
| Glucose | 5,000 |
| AgNO3 | 5 |
| Bacto-Agar | 8,000 |
| adjust pH to 5.8 | |

N6OSM-no2,4-D

| Compound | Amount [mg/L] |
|---|---|
| N6 salt | |
| Casein | 100.0 |
| L-Proline | 700 |
| Mannitol | 36,400 (0.2 M) |
| Sucrose | 20,000 |
| Sorbitol | 36,400 (0.2 M) |
| Bacto-Agar | 15,000 |
| adjust pH to 5.8 | |

MRM1

| Compound | Amount [mg/L] |
|---|---|
| MS salts | |
| MS vitamins | |
| Myoinositol | 100 |
| Sucrose | 6% |
| Bacto-Agar | 9,000 |
| adjust pH to 5.8 | |

MS0

| Compound | Amount [mg/L] |
|---|---|
| MS salts | |
| MS vitamins | |
| Myoinositol | 2,000 |
| Sucrose | 2% |
| Bacto-Agar | 8,000 |
| adjust pH to 5.0 | |

### Example 3: Identification of Successful Editing Events

Two weeks after bombardment, at the end of the type-II callus induction phase, type-II calluses from each bombardment plate were collected. Calluses developed from immature embryos on the same plate were pooled as one sample. Genomic DNA was extracted from each sample using a cetyltrimethylammonium bromide (CTAB)-chloroform-based method and droplet digital PCR (ddPCR) was performed to check for insertion of the DNA fragment at the target genomic locus (CWI2 promoter region).

Insertion junction analysis was performed by sanger sequencing. To this end, sample enrichment for insertion events was carried out by restriction digestion with unique enzymes such as BseX31, Eagl, Fsel, and Sspl that digest the CWI2 promoter region but not the inserted DNA fragment.

### Example 4: Boosting Pol θ-Mediated Transgene Integration by PoIQ Overexpression

In order to increase the efficiency of transgene integration the construct overexpressing ZmPoIQ (SEQ ID NOs: 180 (plasmid)) was co-bombarded with plasmid constructs expressing MAD7 (for example, plasmid SEQ ID NO: 165, MAD7 SEQ ID NO: 166 (CDS) and 167 (PRT)) endonuclease and tDTomato (SEQ ID NO: 168 (CDS) and 169 (PRT)), a regeneration booster protein (for example, plasmid SEQ ID NO: 170, RBP2 CDS: 171, RBP2 Protein SEQ ID NO: 172), two guide RNAs (either GEMT201 (SEQ ID Nos: 173 and 176) with GEMT202 (SEQ ID NO: 174 and 177) or GEMT201 with GEMT248 (SEQ ID Nos: 175 and 178)), that direct the endonuclease to the target site (e.g. the CWI2 promoter, see FIGS. 6 and 7) and the double-stranded DNA insert (SEQ ID NO: 179) into maize immature embryos using biolistic delivery (see example 2). All components were precipitated on the same gold particles. Successful editing events were determined according to the procedure described in example 3.

### Example 5: Generation of Pol θ-Repaired Plants with Targeted Transgene Integration

In addition to analyzing editing events at the embryonic stage, targeted insertion and its efficiency can also be investigated at the plant level. For this purpose, embryonic structures from Example 4 were allowed to regenerate into seedlings. Leaf tissue samples were harvested for genomic DNA isolation, ddPCR, and sequencing analysis. Leaves of the single regenerated plants were collected for DNA extraction. Editing at the target site can be analyzed using ddPCR as described in example 3 and/or using Sanger sequencing.

### Example 6: Combination of Pol θ Overexpression with Agents that Promote Pol θ-Mediated MMEJ/HMEJ Pathway Activity

To further promote the Pole-mediated MMEJ/HMEJ pathway and by that enhancing the efficiency of transgene integration even more, the construct overexpressing ZmPoIQ (SEQ ID NOs: 180 (plasmid)) was co-bombarded with plasmid constructs expressing MAD7 (for example, plasmid SEQ ID NO: 165, MAD7 SEQ ID NO: 166 (CDS) and 167 (PRT)) endonuclease and tDTomato (SEQ ID NO: 168 (CDS) and 169 (PRT)), a regeneration booster protein (for example, plasmid SEQ ID NO: 170, RBP2 CDS: 171, RBP2 Protein SEQ ID NO: 172), two guide RNAs (either GEMT201 (SEQ ID Nos: 173 and 176) with GEMT202 (SEQ ID NO: 174 and 177) or GEMT201 with GEMT248 (SEQ ID Nos: 175 and 178)), that direct the endonuclease to the target site (e.g. the CWI2 promoter, see FIGS. 6 and 7) and the double-stranded DNA insert (SEQ ID NO: 179) and vector constructs expressing proteins that are involved in the MMEJ/HMEJ pathway such as ZmPARP1 (SEQ ID NO: 141 (CDS) and 142 (PRT), encoded by GEMT341 (SEQ ID NO: 181)) and ZmMre11 (SEQ ID NO: 117 (CDS) and 118 (PRT), encoded by GEMT340 (SEQ ID NO: 182)). All components were precipitated on the same gold particles. Successful editing events were determined according to the procedure described in example 3.

### Example 7: Combination of PoIQ Overexpression with Agents that Inhibit Competing DNA Repair Pathways

To further promote Pol theta-mediated transgene integration NHEJ and HDR pathway inhibiting treatments will be performed. Competing NHEJ and HDR pathways can be downregulated using small molecules inhibiting NHEJ and HDR pathway components or by silencing NHEJ and HDR pathway components (e.g. RPA70C (SEQ ID NO: 29 and 30 sqq.), Ku70 (SEQ ID NO: 75 and 76 sqq.), Ku80 (SEQ ID NO: 95 and 96 sqq.), or DANA ligase 4/IV (Lig4) (SEQ ID NO: 53 and 54 sqq.)) on a genomic level. To pursue the latter, plasmids encoding silencing constructs for ZmRPA70C (SEQ ID NO: 183 (plasmid), SEQ ID NO: 184 (siRNA)), ZmKu70 (SEQ ID NO: 185 (plasmid), SEQ ID NO: 186 (siRNA)), ZmKu80 (SEQ ID NO: 187 (plasmid), SEQ ID NO: 188 (siRNA)) and ZmLig4 (SEQ ID NO: 189 (plasmid), SEQ ID NO: 190 (siRNA)) were co-bombarded with construct overexpressing ZmPoIQ (SEQ ID NOs: 180 (plasmid)), plasmid constructs expressing MAD7 (Plasmid SEQ ID NO: 165, MAD7 SEQ ID NO: 166 (CDS) and 167 (PRT)) endonuclease and tDTomato (SEQ ID NO: 168 (CDS) and 169 (PRT)), a regeneration booster protein (Plasmid SEQ ID NO: 170, RBP2 CDS: 171, RBP2 Protein SEQ ID NO: 172), two guide RNAs (either GEMT201 (SEQ ID Nos: 173 and 176) with GEMT202 (SEQ ID NO: 174 and 177) or GEMT201 with GEMT248 (SEQ ID Nos: 175 and 178)), that direct the endonuclease to the target site (e.g. the CWI2 promoter, see FIGS. 6 and 7) and the double-stranded DNA insert (SEQ ID NO: 179) and plasmid constructs expressing proteins that aid Pol θ activity such as ZmPARP1 (SEQ ID NO: 141 (CDS) and 142 (PRT), encoded by GEMT341 (SEQ ID NO: 181)) and ZmMre11 (SEQ ID NO: 117 (CDS) and 118 (PRT), encoded by GEMT340 (SEQ ID NO: 182)). All components were precipitated on the same gold particles. Successful editing events were determined according to the procedure described in example 3.

1. A method for targeted genome modification in a eukaryotic cell, preferably for increasing targeted insertion of a DNA insert at at least one target genomic sequence in a eukaryotic cell, comprising or consisting of the steps:
a) providing at least one eukaryotic cell to be modified;
b) promoting DNA polymerase theta (Pol θ) activity in said cell, wherein the promotion of Pol θ activity increases the DNA insertion efficiency during genome modification;
c) introducing into the at least one eukaryotic cell
(i) at least one genome modification system, preferably a genome editing system comprising at least one site-directed nuclease, nickase or an inactivated nuclease, preferably a nucleic acid guided nuclease, nickase or an inactivated nuclease, or a sequence encoding the same, and optionally at least one guide molecule, or a sequence encoding the same and
(ii) at least one single-stranded or double-stranded DNA insert, or a sequence encoding the same;
d) cultivating the at least one eukaryotic cell under conditions allowing
(i) the promotion of DNA polymerase Pol θ (Pol θ) activity; and
(ii) the activity of the at least one genome editing system and the at least one DNA insert and optionally the at least one guide molecule; and
e) obtaining at least one modified cell comprising the sequence of the DNA insert integrated at or close to the target genomic site; and
f) optionally: obtaining a eukaryotic organism, plant tissue, organ or seed regenerated from the at least one modified cell.

2. The method according to claim 1, wherein promoting of Pol θ is performed by enhancing the cellular level of polymerase Pol θ, or of at least one functional domain thereof.

3. The method according to claim 1, wherein the DNA polymerase Pol θ sequence, or the POLQ sequence encoding the same, is selected from the group consisting of SEQ ID NO: 1 to SEQ ID NO: 26, or a sequence, including a functional domain, having at least at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity thereto.

4. The method according to claim 1, wherein the activity of DNA polymerase Pol θ, or of at least one functional domain thereof, is further promoted by modulating, preferably silencing, components of at least one competing DNA repair pathway, wherein the component belongs to the NHEJ or HDR repair pathway, or the sequence encoding the same, is selected from the group consisting of SEQ ID NO: 27 to SEQ ID NO: 114, or a sequence having at least at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity thereto.

5. The method according to claim 1, wherein the promotion of MMEJ/HMIEJ pathway is achieved by co-expressing and/or modulating at least one MRE11 and/or at least one PARP1 protein, wherein the MRE11 and/or PARP1 protein, or the sequence encoding the same, is selected from the group consisting of SEQ ID NO: 115 to SEQ ID NO: 162 or a sequence having at least at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity thereto.

6. The method of claim 1, wherein at least one site-directed nuclease, nickase or inactivated nuclease, or a sequence encoding the same, is selected from the group consisting of a CRISPR/Cas system, preferably from a CRISPR/Cas12a or a CRISPR/Cas12b system, including a CRISPR/MAD7 system, a CRISPR/Cfp1 system, or a CRISPR/MAD2 system, from a CRISPR/Cas9 system, a CRISPR/CasX system, a CRISPR/CasY system, a CRISPR/Cas13 system, a CRISPR/Csm system, a zinc finger nuclease system, a transcription activator-like nuclease system, a meganuclease system, and any combination, variant, or catalytically active fragment thereof.

7. The method according to claim 1, wherein Pol θ activity, or the at least one functional domain thereof, promotes targeted genome modification by acting on an endogenous micro-homology mediated end joining (MMEJ) and/or a homology-mediated end-joining (HMEJ) pathway.

8. The method according to claim 1, wherein the eukaryotic cell is a plant cell and wherein the method additionally comprises a step g) of screening for at least one plant tissue, organ, plant or seed regenerated from the at least or modified cell in the TO and/or T1 generation carrying the DNA insert.

9. The method according to claim 1, wherein the at least one eukaryotic cell is selected from a plant cell, and a mammalian cell.

10. The method according to claim 1, wherein the at least one eukaryotic cell is a plant cell.

11. The method according to claim 10, wherein the plant cell is a plant cell selected from the group consisting of Hordeum, Sorghum, Saccharum, Zea, Setaria, Oryza, Triticum, Secale, Triticale, Malus, Brachypodium, Aegilops, Daucus, Beta, Eucalyptus, Nicotiana, Solanum, Coffea, Vitis, Erythrante, Genlisea, Cucumis, Marus, Arabidopsis, Crucihimalaya, Cardamine, Lepidium, Capsella, Olmarabidopsis, Arabis, Brassica, Eruca, Raphanus, Citrus, Jatropha, Populus, Medicago, Cicer, Cajanus, Phaseolus, Glycine, Gossypium, Astragalus, Lotus, Torenia, Allium, Spinacia and Helianthus, preferably, the plant or plant cell originates from a species selected from the group consisting of Hordeum vulgare, Hordeum bulbusom, Sorghum bicolor, Saccharum officinarium, Zea spp., including Zea mays, Setaria italica, Oryza minuta, Oryza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta spp., including Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinfera, Erythrante guttata, Genlisea aurea, Cucumis sativus, Marus notabilis, Arabidopsis arenosa, Arabidopsis lyrata, Arabidopsis thaliana, Crucihimalaya himalaica, Crucihimalaya wallichii, Cardamine nexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanfolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Allium tuberosum, Helianthus annuus, Helianthus tuberosus and Spinacia oleracea.

12. The method according to claim 1, wherein the method additionally comprises the step of introducing or applying at least one regeneration booster, or a sequence encoding the same, or a regeneration booster chemical.

13. The method according to claim 12, wherein the regeneration booster is selected from the group consisting of BBM, WUS, WOX, RKD4, RKD2, GRF, LEC, and variants thereof, or a sequence encoding the same.

14. The method according to claim 12, wherein the regeneration booster comprises at least one of an RBP and/or at least one PLT, preferably wherein the regeneration booster comprises at least one of an RBP, wherein the at least one regeneration booster sequence is individually selected from any one of SEQ ID NOs: 171, 195 to 201, and 209 to 211 or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or an active fragment thereof, or wherein the at least one regeneration booster sequence is encoded by a sequence individually selected from any one of SEQ ID NO: 172, 202 to 208, and 212 to 214 or a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity thereto.

15. The method according to claim 14, wherein at least one first RBP or PLT, or a sequence encoding the same, preferably at least one RBP, most preferably RBP2, or the sequence encoding the same, is provided and wherein at least one further regeneration booster is provided selected from:
(i) at least one further RBP and/or PLT, or the sequence encoding the same, or a variant thereof,
(ii) at least one BBM, or the sequence encoding the same, or a variant thereof,
(iii) at least one WOX, including WUS1, WUS2, or WOX5, or the sequence encoding the same, or a variant thereof,
(iv) at least one RKD4 or RKD2, including wheat RKD4, or the sequence encoding the same, or a variant thereof,
(v) at least one GRF, including Zea mays GRF5 and Zea mays GRF1/TOW, or the sequence encoding the same, or a variant thereof, and/or
(vi) at least one LEC sequence, including LEC1 and LEC2, or the sequence encoding the same, or a variant thereof,
and wherein the at least one second regeneration booster, or a sequence encoding the same, is different to the first regeneration booster.

16. A genetically modified cell, tissue, organ, or material, including a seed, obtainable by a method according to claim 1.

17. The genetically modified cell according to claim 14, wherein the cell, tissue, organ, or material, including a seed, is a plant cell, tissue, organ, or material, including a seed.

18. An expression construct assembly, comprising:
(i) at least one vector encoding at least one Pol θ sequence, or at least one functional domain thereof, and optionally comprising a construct encoding silencing components of at least one MMEJ/HMEJ competing repair pathway, and optionally a protein promoting the MMEJ/HMEJ pathway, and
(ii) at least one vector encoding a gene encoding at least one genome editing system, preferably wherein the genome editing system is as defined in claim 1, optionally comprising at least one vector encoding at least one guide molecule as defined in claim 1; and
(iii) at least one vector encoding at least one DNA insert, or at least one single-stranded or double-stranded DNA insert as defined in claim 1; and
(iv) optionally: at least one vector encoding at least one regeneration booster, preferably wherein the regeneration booster comprises (a) BBM, WUS, WOX, RKD4, RKD2, GRF, LEC, and variants thereof, or a sequence encoding the same, or (b) at least one of an RBP and/or at least one PLT, preferably wherein the regeneration booster comprises at least one of an RBP, wherein the at least one regeneration booster sequence is individually selected from any one of SEQ ID NOs: 171, 195 to 201, and 209 to 211 or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or an active fragment thereof, or wherein the at least one regeneration booster sequence is encoded by a sequence individually selected from any one of SEQ ID NO: 172, 202 to 208, and 212 to 214 or a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity thereto;
wherein (i), (ii), (iii), and/or (iv) are encoded on the same, or on different vectors,
wherein the DNA polymerase Pol θ sequence, or the POLQ sequence encoding the same, is selected from the group consisting of SEQ ID NO: 1 to SEQ ID NO: 26, or a sequence, including a functional domain, having at least at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity thereto,
wherein the construct encoding silencing components of at least one MMEJ/HMEJ competing repair pathway, belongs to the NHEJ or HDR repair pathway, and the construct or the sequence encoding the same, is selected from the group consisting of SEQ ID NO: 27 to SEQ ID NO: 114, or a sequence having at least at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity thereto, and
wherein the protein promoting the MMEJ/HMEJ pathway, or the sequence encoding the same, is selected from the group consisting of SEQ ID NO: 115 to SEQ ID NO: 162 or a sequence having at least at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity thereto.

19. The expression construct assembly according to claim 18, wherein at least one vector of the assembly further comprises a nucleic acid sequence encoding at least one marker.

20. The expression construct assembly according to claim 18, wherein the expression construct assembly comprises at least one construct or sequence individually selected from any one of SEQ ID NOs: 163 to 165, 170, 173 to 175, and 179 to 183, 185, 187 and 189.
